SEQUENCE LISTING (12 sequences)

SEQ ID NO: 1 - protein, 431 aa, Homo sapiens
  OP-1
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 2 - protein, 96 aa, Artificial Sequence
  Description of Artificial Sequence Synthetic amino acid sequence COP-5
LYVDFSDVGWDDWIVAPPGYQAFYCHGECPFPLADHFNSTNHAVVQTLVNSVNSKIPKACCVPTELSAISMLYLDENEKVVLKYNQEMVVEGCGCR

SEQ ID NO: 3 - protein, 96 aa, Artificial Sequence
  Description of Artificial Sequence Synthetic amino acid sequence COP-7
LYVDFSDVGWNDWIVAPPGYHAFYCHGECPFPLADHLNSTNHAVVQTLVNSVNSKIPKACCVPTELSAISMLYLDENEKVVLKYNQEMVVEGCGCR

SEQ ID NO: 4 - protein, 102 aa, Artificial Sequence
  MOD_RES (1)..(102): Description of Artificial Sequence  OPX Xaa is independently selected from a group of one or more specified amino acids as defined in the specification
CXXHELYVXFXDLGWXDWXIAPXGYXAYYCEGECXFPLXSXMNATNHAIXQXLVHXXXPXXVPKXCCAPTXLXAXSVLYXDXSXNVXLXKXRNMVVXACGCH

SEQ ID NO: 5 - protein, 97 aa, Artificial Sequence
  MOD_RES (1)..(97): Description of Artificial Sequence Generic Sequence 7 Xaa is independently selected from a group of one or more specified amino acids as defined in the specification
LXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 6 - protein, 102 aa, Artificial Sequence
  MOD_RES (1)..(102): Description of Artificial Sequence Generic Sequence 8 Xaa is independently selected from a group of one or more specified amino acids as defined in the specification
CXXXXLXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 7 - protein, 97 aa, Artificial Sequence
  MOD_RES (1)..(97): Description of Artificial Sequence Generic Sequence 9 Xaa is independently selected from a group of one or more specified amino acids as defined in the specification
XXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 8 - protein, 102 aa, Artificial Sequence
  MOD_RES (1)..(102): Description of Artificial Sequence Generic Sequence 10 Xaa is independently selected from a group of one or more specified amino acids as defined in the specification
CXXXXXXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 9 - protein, 5 aa, Artificial Sequence
  Description of Artificial Sequence Consensus sequence
CXXXX

SEQ ID NO: 10 - DNA, 1822 bp, Homo sapiens
  CDS (49)..(1341): OP-1
ggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatgaaaaaaaaaaaaaaaaaa

SEQ ID NO: 11 - DNA, 1372 bp, rat
  Collagen promoter
aatataagccacttttcttgggggacgacaaatgaccctttcctgattgcagaggtggggaacaatggctgagattttcagcaaagaagcgaggacatgaggagtagccttcaaataaagtcactcagctaccaaaaacaagtttctgccacacaccgagttacctaggtgtccccagaccagatccaagtacagtaaggaaagcaggttctctacagagagaacacggctctatggccaatgccttctacctgctctttctggattgatactgctacctaagagggcctctaaccaattcctggctgtagccacagctgacacaagacctttttctaagacatccctggtcacaggcctcctgtagcaaattccagccctgggatggaggtggtcaggaaagagtttatacaagaagacccaggccacagctttaaggactcagaaacccccctgcccacacggctgcccatcataacgcagaaggtttcttctggaaggacaaggatgtcaaacttctccccaagcctaatcctcagagatgtctccctctgttacacctggggctggagaaaggtgggtctttcatggagccacattcatggcagaacagatagccaccccactcctttcaaacaaccacatatctgactcttagtatctgtgaagagatgtctaatttgttcccaaatattcctaccctgcatacctgggcccacaccatgaggtattctcctccctctaacagtcacatctgcttagctgcctggttcttcggatttggagagatgcttgcctaacttattcttccttaggtcttcccaaggatgccagaaagactatgagacatggccaagaggaccttttcccaattgtgcctgacactgaaccctttgtaatgttccccaactcagattcccaattctacatccttctgatttgaggtcccagaaggaaagtgcaaggggcatcccctacccacaatcagtatatcgaggcccagccacactcagtgatagcacctctggcccatgtagatctgggggacaagggtggcagaattgcaaaggggggagggggctgggtggactcctttcccttcctttccctcctcccccctcttcgttccaaattgggggccgggccaggcagttctgattggctgggggccgggctgctggctccccctctccaagaggcagggttcctcccagccctcctccatcaggatggtataaaaggggcccaggccagtcgtcggagcagacgggagtttcacctccggacggagcaggaggcacacggagtgaggccacgcatgagccgaagctaaccccccaccccagccgcaaagagtctacatgtctagggt

SEQ ID NO: 12 - DNA, 757 bp, rat
  Collagen promoter with deletion
aatataagccacttttcttgggggacgacaaatgaccctttcctgattgcagaggtggggaacaatggctgagattttcagcaaagaagcgaggacatgaggagtagccttcaaataaagtcactcagctaccaaaaacaagtttctgccacacaccgagttacctaggtgtccccagaccagatccaagtacagtaaggaaagcaggttctctacagagagaacacggctctatggccaagaggaccttttcccaattgtgcctgacactgaaccctttgtaatgttccccaactcagattcccaattctacatccttctgatttgaggtcccagaaggaaagtgcaaggggcatcccctacccacaatcagtatatcgaggcccagccacactcagtgatagcacctctggcccatgtagatctgggggacaagggtggcagaattgcaaaggggggagggggctgggtggactcctttcccttcctttccctcctcccccctcttcgttccaaattgggggccgggccaggcagttctgattggctgggggccgggctgctggctccccctctccaagaggcagggttcctcccagccctcctccatcaggatggtataaaaggggcccaggccagtcgtcggagcagacgggagtttcacctccggacggagcaggaggcacacggagtgaggccacgcatgagccgaagctaaccccccaccccagccgcaaagagtctacatgtctagggt

(19) United States
(12) Patent Application Publication
(10) Pub. No.: US 2005/0152883 A1
(43) Pub. Date: Jul. 14, 2005

(54) Compositions and methods for ligament growth and repair

(76) Inventors: John C. Lee, San Antonio, TX (US); Lee-Chuan C. Yeh, San Antonio, TX (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 11/021,902

(22) Filed: Dec. 21, 2004

Related U.S. Application Data
(63) Continuation of application No. PCT/US03/21697, filed on Jul. 9, 2003.
(60) Provisional application No. 60/395,110, filed on Jul. 9, 2002.

(51) Int. Cl. A61K 48/00; A61K 38/17

(52) U.S. Cl. 424/93.7; 424/93.21; 514/12

(57) ABSTRACT

The present invention provides methods and compositions for treating and repairing ligament defects using a bone morphogenic protein. The present invention provides methods of treating ligament defects, repairing ligament defects, forming ligament tissue, regenerating ligament tissue, and promoting growth of ligament tissue by transplanting into a patient in need thereof ligament cells cultured ex-vivo.

### FIELD OF THE INVENTION

The present invention relates to orthopaedic tissue transplantation. More particularly, it relates to methods of treating, repairing or regenerating ligament tissue by transplanting into a defect site ligament cells cultured ex-vivo.

### BACKGROUND OF THE INVENTION

Ligament serves to connect bone or cartilage across joints. Ligaments are composed of substantially parallel bundles of white fibrous tissue. They are pliant and flexible to allow substantially complete freedom of movement, but are inextensile to prevent over-extension of the interacting bones in the joint. Defects in ligament tissue, due to disease or damage, can result in pain, instability, and loss of movement. Injuries to the medial collateral ligament (“MCL”) and anterior cruciate ligament (“ACL”) are particularly common.

The ACL of the knee connects the bottom of the thigh bone (femur) and the top of the shin bone (tibia). The ACL acts to resist anterior displacement of the tibia from the femur. It also acts to resist hyperextension of the knee. The MCL is located on the inner side of the knee and connects the femur to the tibia. The MCL prevents the knee joint from medial instability thus preventing the leg from moving outwards on the thigh bone.

Repair of ligament is a complex process involving cellular proliferation and migration, as well as synthesis and deposition of ligament cellular components. Growth factors such as basic fibroblast growth factor (bFGF), platelet derived growth factor-B (PDGF-B), insulin growth factor-I and -II (IGF-I and IGF-II) and transforming growth factor-β (TGF-β) have been shown to stimulate the synthesis of extracellular protein molecules and cell proliferation of ligamentous cells (see Benjamin et al., Int. Rev. Cytol., 196: 85-130 (2000); Woo et al., Clin. Orthop., S: 312-23 (1999); Koyabashi et al. Knee Surg. Sports Traumatol Arthrosc., 5: 189-94 (1997); Maurai et al., J. Orthop. Res., 15: 18-23 (1997); Schmidt et al., J. Orthop. Res., 13: 184-90 (1995); Woo et al., Med. Bio. Eng. Comput., 36: 359-64 (1998); Scherping et al., Connect. Tissue Res., 36: 1-8 (1997); Spindler et al., J. Orthop. Res., 14: 542-46 (1996); Abrahamsson, J. Orthop. Res., 15: 256-62 (1997); Murphy et al., Am. J. Vet. Res., 58: 103-09 (1997); Natsu-ume et al., J. Orthop. Res., 15: 837-43 (1997); Spindler et al., J. Orthop. Res., 20: 318-24 (2002); and Kuroda et al., Knee Surg. Sports Traumatol Arthrosc., 8: 120-26 (2000)).

Bone morphogenic proteins have also been demonstrated to play a role in ligament and tendon formation. For example, GDF-5 (BMP-14), GDF-6 (BMP-13), and GDF-7 (BMP-12) have been shown to induce tendon and ligament formation when implanted at ectopic sites in vivo (see, e.g., Aspenberg et al., Acta Orthop. Scand., 70: 51-54 (1999), Forslund et al., Med. Sci. Sports Exerc., 33: 685-75 (2001), Tashiro et al. Orthop. Res. Soc., 24: 301 (1999), and Wolfman et al., J. Clin. Invest., 100: 321-30 (1997)).

Ligament tissue is substantially devoid of blood vessels and has little or no self-regenerative properties. Ligament damage is sometimes repaired by non-surgical rehabilitation. However, surgical repair is required when rehabilitation is insufficient to heal the damage. Methods of surgical repair of torn or damaged ligament tissue have been limited to the use of autogenous grafts: or synthetic materials that are surgically attached to the articular extremities of the bones. However, some patients require multiple operations due to graft failure.

Thus, there remains a need for new methods and compositions for treating and repairing ligament defects. There also remains a need for methods and compositions of forming and/or regenerating ligament tissue and/or promoting growth of ligament tissue.

### SUMMARY OF INVENTION

The present invention provides methods and compositions for treating and repairing ligament defects using a bone morphogenic protein. The present invention provides methods of treating ligament defects, repairing ligament defects, forming ligament tissue, regenerating ligament tissue, and promoting growth of ligament tissue by transplanting into a patient in need thereof ligament cells cultured ex-vivo and administering a bone morphogenic protein. The methods of this invention comprise the following steps:
(a) isolating ligament cells;
(b) culturing the ligament cells ex-vivo;
(c) recovering the cultured ligament cells; and
(d) implanting the recovered ligament cells into the patient.

The invention also provides compositions for treating, repairing and regenerating ligament tissue as well as: compositions for forming and promoting ligament tissue comprising cultured ligament cells and a bone morphogenic protein.

### BRIEF DESCRIPTION OF THE DRAWINGS

FIG. 1 depicts cell morphology of primary cultures of rat MCL cells. Cells were cultured in DMEM/F12 medium with 10% FBS. Media were changed every 3 days. Cell morphology was monitored as a function of time with an Olympus CK2 inverted microscope equipped with a CCD camera. Representative images (phase contrast with 100× magnification) of cells of passage 1 (FIG. 1A) and passage 2 (FIG. 1B) are presented.

FIG. 2 is a graphical representation of the effect of OP-1 on rat MCL cell proliferation. MCL cells were grown to confluency and treated with the various amounts of OP-1 for 24 h. Cell proliferation was determined by a colometric assay. Values were normalized to the vehicle control (as 1) and represent the mean +/−SEM of seven independent measurements.

FIG. 3 is a graphical representation of the effect of OP-1 on alkaline phosphatase (“AP”) activity in primary cultures of rat MCL cells. MCL cells were grown to confluency and treated with various concentrations of OP-1 (50, 100, 200, 300, 400, and 500 ng/ml). Total AP activity in the cell lysate was measured after 48 h. Values were normalized to the solvent control (as 1) and represent the mean +/−SEM of three different determinations on two different MCL cell preparations.

FIG. 4 depicts Six1 and scleraxis mRNA expression levels in long-term cultures of control: and OP-1-treated rat MCL cells. FIG. 4A is a Northern blot of Six1 and scleraxis mRNA. Confluent MCL cells were treated with solvent vehicle or 200 ng/ml of OP-1 for different durations. Total RNA was isolated on the designated day, denatured, resolved on 1% agarose gel containing formaldehyde, and transferred onto a Nytran Plus membrane. The blots were hybridized with the cDNA probes for Six1, scleraxis, or the oligonucleotide probe for 18S rRNA. After washing under appropriate conditions, the blots were exposed to a PhosphorImage screen. FIG. 4B is a quantitative analysis of the Six1 mRNA level in MCL cells depicted in FIG. 4A. The intensity of the hybridized RNA shown in FIG. 4A was analyzed by ImageQuant software. The mRNA level was normalized to the 18S rRNA level. The normalized mRNA level was then compared to the control value on day 0 (the day treatment began) as 1. FIG. 4C is a quantitative analysis of the scleraxis mRNA level in MCL cells depicted in FIG. 4A. Values represent the mean +/−SEM of two independent measurements.

FIG. 5 is a graphical representation of the effect of OP-1 on Run2x/Cbfa1 mRNA expression in long-term cultures of rat MCL cells as measured by Northern blot analysis. Confluent MCL cells were treated as described in FIG. 4. Blots were probed with the cDNA probe for Run2x/Cbfa1. Values represent the mean +/−SEM of two independent measurements.

FIG. 6 is a graphical representation of the effect of OP-1 on the steady-state mRNA level of type I collagen in long-term cultures of rat MCL cells as measured by Northern blot analysis. Confluent MCL cells were treated as described in FIG. 4. Blots were probed with the cDNA probe for type I collagen. Values represent the mean +/−SEM of two independent measurements.

FIG. 7 is a graphical representation of the effect of OP-1 on the promoter activity of type I collagen transiently transfected into rat MCL cells. Primary cultures of rat MCL cells were transfected with the type I collagen promoter constructs described in Example 4 and treated with solvent, 50 or 200 ng/ml of Op-1 for six days. The luciferase activity was then measured and normalized to the β-galactosidase activity using the Dual assay kit (Tropix, Bedford, Mass.). Values represent the mean +/−SEM of two independent determinations.

FIG. 8 is a representative Northern blot of −25 ActR-I, BMPR-IA, BMPR-IB, BMPR-II, and 18S in long-term cultures of control and OP-1-treated rat MCL cells. MCL cells were treated with 200 ng/ml of OP-1 for the indicated time. Media were refreshed every three days. Total RNA was isolated and processed as described in FIG. 4. The blots were hybridized with the cDNA probes for ActR-I BMPR-IA, BMPR-IB, BMPR-II, respectively, or the olglionucleotide probe for 18S rRNA.

FIG. 9 is a the graphical representation of the Northern blots depicted in FIG. 8. The results shown in FIG. 8 were quantified as described in FIG. 4. Values represent the mean +/−SEM of two independent measurements.

FIG. 10 is a representative RNase protection analysis blot demonstrating BMP-1, -2, -4, and -6 mRNA expression in control and OP-1-treated rat MCL cells. Confluent cultures were treated with vehicle or 200 ng/ml of OP-1 for the designated days. Total RNA was isolated using the TRI reagent. 20 μg of total RNA was used for the measurement of BMP mRNA in the RNase protection assay. The protected RNA fragments were fractionated on 5% polyalcrylamide gels containing 8M urea and detected by PhosphorImaging. Positions of the labeled probes for the different BMPs and the two housekeeping gene controls (ribosomal protein L32 and GAPDH) are on the left of the image. The protected fragments are indicated on the right.

FIG. 11 is a graphical representation of the RNase protection analysis depicted in FIG. 10. The intensity of the protected fragments as shown in FIG. 10 was analyzed and quantified using the ImageQuant software. Values represent mean +/−SEM from two to three different determinations.

FIG. 12 is a representative RNase protection analysis blot demonstrating GDF-1, -3, -5, -6, and -8 mRNA expression in control and OP-1-treated rat MCL cells. Confluent cultures were treated with vehicle or 200 ng/ml of OP-1 for the designated days. Total RNA was analyzed as described in FIG. 10. Positions of labeled probes for the different GDFs and the two housekeeping gene controls (ribosomal protein L32 and the GAPDH) are on the left of the image. The protected fragments are indicated on the right.

FIG. 13 is a graphical representation of the RNase protection analysis depicted in FIG. 12. The intensity of the protected fragments as shown in FIG. 12 was analyzed and quantified using the ImageQuant software. Values represent mean +/−SEM from two to three different determinations.

### DETAILED DESCRIPTION OF THE INVENTION

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

In order to further define the invention, the following terms and definitions are provided herein.

The term “ligament” refers to substantially parallel bundles of connective tissue that attach bones or cartilage across joints. Examples of ligament include but are not limited to ACL and MCL.

The term “ligament cell” refers to any cell which when exposed to the appropriate stimulus or stimuli, is capable of expressing and secreting components characteristic of ligament tissue. Ligament cells include cells at varying stages of differentiation. Ligament cells as defined herein may be capable of proliferation and may be induced to differentiate upon exposure to the appropriate stimulus or stimuli. Ligament cells may be isolated directly from pre-existing ligament tissue or from mesenchymal stem cells in the bone marrow.

The term “defect” or “defect site”, refers to a disruption of a ligament requiring repair. A defect can assume the configuration of a “void”, which is understood to mean a three-dimensional defect such as, for example, a gap, cavity, hole or other substantial disruption in the structural integrity of a ligament. A defect can also be a detachment of the ligament from its point of attachment to the bone or cartilage. In certain embodiments, the defect is such that it is incapable of endogenous or spontaneous repair. A defect can be the result of accident, disease, and/or surgical manipulation.

The term “repair” refers to new ligament formation which is sufficient to at least partially fill the void or structural discontinuity at the defect. Repair does not, however, mean, or otherwise necessitate, a process of complete healing or a treatment which is 100% effective at restoring a defect to its pre-defect physiological/structural/mechanical state.

The term “therapeutically effective amount” refers to an amount effective to repair, regenerate, promote, or form ligament tissue.

The term “patient” refers to an animal, including a mammal (e.g., a human).

The term “morphogenic protein” refers to a protein having morphogenic activity. Preferably a morphogenic protein of this invention comprises at least one polypeptide belonging to the BMP protein family. Morphogenic proteins include osteogenic proteins. Morphogenic proteins may be capable of inducing progenitor cells to proliferate and/or to initiate differentiation pathways that lead to cartilage, bone, tendon, ligament or other types of tissue formation depending on local environmental cues, and thus morphogenic proteins may behave differently in different surroundings. For example, a morphogenic protein may induce bone tissue at one treatment site and ligament tissue at a different treatment site.

The term “bone morphogenic protein (BMP)” refers to a protein belonging to the BMP family of the TGF-β superfamily of proteins (BMP family) based on DNA and amino acid sequence homology. A protein belongs to the BMP family according to this invention when it has at least 50% amino acid sequence identity with at least one known BMP family member within the conserved C-terminal cysteine-rich domain which characterizes the BMP protein family. Preferably, the protein has at least 70% amino acid sequence identity with at least one known BMP family member within the conserved C-terminal cystein rich domain. Members of the BMP family may have less than 50% DNA or amino acid sequence identity overall.

The term “amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence. Certain particularly preferred morphogenic polypeptides share at least 60%, and preferably 70% amino acid sequence identity with the C-terminal 102-106 amino acids, defining the conserved seven-cysteine domain of human OP-1 and related proteins.

Amino acid sequence homology can be determined by methods well known in the art. For instance, to determine the percent homology of a candidate amino acid sequence to the sequence of the seven-cysteine domain, the two sequences are first aligned. The alignment can be made with, e.g., the dynamic programming algorithm described in Needleman et al., J. Mol. Biol., 48, pp. 443 (1970), and the Align Program, a commercial software package produced by DNAstar, Inc. The teachings by both sources are incorporated by reference herein. An initial alignment can be refined by comparison to a multi-sequence alignment of a family of related proteins. Once the alignment is made and refined, a percent homology score is calculated. The aligned amino acid residues of the two sequences are compared sequentially for their similarity to each other: Similarity factors include similar size, shape and electrical charge. One particularly preferred method of determining amino acid similarities is the PAM250 matrix described in Dayhoff et al., Atlas of Protein Sequence and Structure, 5, pp. 345-352 (1978 & Supp.), which is incorporated herein by reference. A similarity score is first calculated as the sum of the aligned pairwise amino acid similarity scores. Insertions and deletions are ignored for the purposes of percent homology and identity. Accordingly, gap penalties are not used in this calculation. The raw score is then normalized by dividing it by the geometric mean of the scores of the candidate sequence and the seven-cysteine domain. The geometric mean is the square root of the product of these scores. The normalized raw score is the percent homology.

The term “conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., supra. Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. The term “conservative variant” or “conservative variation” also includes the use of a substituting amino acid residue in place of an amino acid residue in a given parent amino acid sequence, where antibodies specific for the parent sequence are also specific for, i.e., “cross-react” or “immuno-react” with, the resulting substituted polypeptide sequence.

The term “osteogenic protein (OP)” refers to a morphogenic protein that is capable of inducing a progenitor cell to form cartilage and/or bone. The bone may be intramembranous bone or endochondral bone. Most osteogenic proteins are members of the BMP protein family and are thus also BMPs. As described elsewhere herein, the class of proteins is typified by human osteogenic protein (hOP-1). Other osteogenic proteins useful in the practice of the invention include osteogenically active forms of OP-1, OP-2, OP-3, BMP-2, BMP-3, BMP-4, BMP-5, BMP-6, BMP-8, BMP-9, BMP-10, DPP, Vg1, Vgr-1, 60A protein, GDF-1, GDF-2, GDF-3, GDF-5, GDF-6, GDF-7, GDF-8, GDF-9, GDF-10, GDF-11, GDF-12, BMP-11, BMP-15, BMP-16, UNIVIN, NODAL, SCREW, ADMP or NEURAL and amino acid sequence variants thereof. Osteogenic proteins suitable for use with applicants' invention can be identified by means of routine experimentation using the art-recognized bioassay described by Reddi and Sampath (Sampath et al., Proc. Natl. Acad. Sci., 84, pp. 7109-13, incorporated herein by reference).

Proteins useful in this invention include eukaryotic proteins identified as osteogenic proteins (see U.S. Pat. No. 5,011,691, incorporated herein by reference), such as the OP-1, OP-2, OP-3 and CBMP-2 proteins, as well as amino acid sequence-related proteins, such as DPP (from Drosophila), Vg1 (from Xenopus), Vgr-1 (from mouse), GDF-1 (from humans, see Lee, PNAS, 88, pp. 4250-4254 (1991)), 60A (from Drosophila, see Wharton et al. PNAS, 8.8, pp. 9214-9218 (1991)), dorsalin-1 (from chick, see Basler et al. Cell 73, pp. 687-702 (1993) and GenBank accession number L12032), GDF-5 (from mouse, see Storm et al. Nature, 368, pp. 639-643 (1994)), GDF-6 and GDF-7. The teachings of the above references are incorporated herein by reference. BMP-3 is also preferred. Additional useful proteins include biosynthetic morphogenic constructs disclosed in U.S. Pat. No. 5,011,691, incorporated herein by reference, e.g., COP-1, COP-3, COP-4, COP-5, COP-7 and COP-16, as well as other proteins known in the art. Still other proteins include osteogenically active forms of BMP-3b (see Takao, et al. Biochem. Biophys. Res. Comm., 219, pp. 656-662 (1996)). BMP-9 (see WO95/33830), BMP-15 (see WO96/35710), BMP-12 (see WO95/16035), CDMP-1 (see WO 94/12814), CDMP-2 (see WO94/12814), BMP-10 (see WO94/26893), GDF-1 (see WO92/00382), GDF-10 (see WO95/10539), GDF-3 (see WO94/15965) and GDF-7 (see WO95/01802). The teachings of the above references are incorporated herein by reference.

Methods and Compositions of Ligament Growth and Repair

The methods and compositions of this invention may be used for ligament growth and repair in a patient. The methods may be used instead of surgical procedures, or in conjunction with surgical procedures to repair ligament. For example, the methods of this invention may be used to aid attachment of surgically implanted graft tissue.

In some embodiments, the invention provides a method for treating ligament defects in a patient, comprising the steps of: (a) isolating ligament cells; (b) culturing the ligament cells ex-vivo; (c) recovering the cultured ligament cells; and (d) implanting the cultured ligament cells into the patient.

In some embodiments, the invention provides a method of repairing ligament defects in a patient comprising the steps of: (a) isolating ligament cells; (b) culturing the ligament cells ex-vivo; (c) recovering the cultured ligament cells; and (d) implanting the cultured ligament cells into the patient.

In some embodiments, the invention provides a method of regenerating ligament tissue in a patient, comprising the steps of: (a) isolating ligament cells; (b) culturing the ligament cells ex-vivo; (c) recovering the cultured ligament cells; and (d) implanting the cultured ligament cells into the patient.

In some embodiments, the invention provides a method of forming ligament tissue in a patient, comprising the steps of: (a) isolating ligament cells; (b) culturing the ligament cells ex-vivo; (c) recovering the cultured ligament cells; and (d) implanting the cultured ligament cells into the patient.

In some embodiments, the invention provides a method of promoting ligament tissue formation in a patient, comprising the steps of: (a) isolating ligament cells; (b) culturing the ligament cells ex-vivo; (c) recovering the cultured ligament cells; and (d) implanting the cultured ligament cells into the patient.

Ligament cells may be isolated from any tissue containing ligament cells. Ligament cells may be isolated directly from pre-existing ligament tissue (e.g. ACL or MCL). Ligament tissue may also be isolated from mesenchymal stem cells in the bone marrow. Ligament tissue may be obtained, for example, by surgical excision, from the patient into whom the ligament cells are to be implanted, or may be obtained from another patient.

In some embodiments, the isolated ligament cells are resuspended in culture medium under conditions effective to maintain their ability to express and secrete components characteristic of ligament tissue. In some embodiments, the ligament cells are resuspended in culture medium under conditions effective to allow the cells to differentiate. The culture medium may further comprise stimulatory agents including but not limited to fetal bovine serum, exogenously added growth factors (e.g., bFGF, PDGF, IGF-I, IGF-II, TGF-β, VEGF, IL-6 in combination with its soluble IL-6 receptor, LIM Mineralization Protein-1), hormones (PTH, insulin, vitamin D), gap junction proteins (e.g., connexin), bone morphogenic proteins (see infra) and/or other agents (e.g., norepinephrine) or any combinations thererof. In some embodiments, the bone morphogenic protein is selected from the group consisting of OP-1, OP-2, OP-3, BMP-2, BMP-3, BMP-4, BMP-5, BMP-6, BMP-8, BMP-9, BMP-10, BMP-11, BMP-15, BMP-16, DPP, Vg1, Vgr-1, 60A protein, GDF-1, GDF-2, GDF-3, GDF-5, GDF-6, GDF-7, GDF-8, GDF-9, GDF-10, GDF-11, GDF-12, NODAL, UNIVIN, SCREW, ADMP, NEURAL, and amino acid sequence variants thereof.

In some embodiments, the ligament cells are transfected with DNA encoding the growth factors and/or bone morphogenic proteins. In a preferred embodiment the ligament cells are transfected with a nucleic acid sequence encoding OP-1 (SEQ. ID NO:10). In some embodiments, the growth factors and bone morphogenic proteins are constitutively expressed. In other embodiments, the expression of the growth factors and/or bone morphogenic proteins is inducible. Methods of transfecting the ligament cells with the desired DNA and expressing the corresponding proteins are well known to the skilled worker (see, e.g., Molecular Cloning A Laboratory Manual, 2^{nd} Ed., ed. by Sambrook et al. (Cold Spring Harbor Laboratory Press 1989) and Current Protocols in Molecular Biology, ed. by Ausubel et al. (Greene Publishing and Wiley Interscience, New York 1998)). One of ordinary skill in the art will also appreciate that other agents may be added to the culture medium to maintain the ligament cells in culture.

In some embodiments, the ligament cells are cultured under conditions that would allow the production of a cell-associated matrix similar to that present in vivo. In some embodiments the ligament cell-associated matrix includes but is not limited to type 1 collagen, elastin, decorin, aggrecan or any combinations thereof.

The cultured ligament cells are recovered from the culture medium using methods well known in the art. One such method includes removing the culture medium and detaching the ligament cells from the culture plates, resuspending the ligament cells in buffer or medium, centrifuging the cells and removing the buffer or medium and resuspending the cells in a buffer or solution appropriate for implantation into a patient. In some embodiments, the cells may be removed from the culture plates by physically scraping them off the plates with a rubber policeman. In some embodiments, the cells may be recovered by digesting the cells with a solution of trypsin-EDTA at room temperature, inhibiting the trypsin activity with serum, and briefly centrifuging the cells at low speed.

In some embodiments the recovered ligament cells comprise ligament cell-associated matrix.

The recovered ligament cells are implanted into the patient at the defect site or the site where it is desired to regenerate or form ligament tissue, or promote its growth. In some embodiments, the implanted ligament cells are transfected with a nucleic acid sequence encoding a bone morphogenic protein and/or a growth factor as described herein. In other embodiments, the cells are untransfected. The cells may be implanted using recognized methods in the art. These include but are not limited to the injection into the defect site or packing cells into the defect site.

In some embodiments, following implantation of the ligament cells, a morphogenic protein may be administered to the patient. The morphogenic protein may be formulated as a pharmaceutical composition. The morphogenic protein may also be implanted with a carrier as described herein (see infra). In some embodiments, the morphogenic protein is administered locally to the defect site or the site where ligament formation/regeneration or repair is desired. In some embodiments, the morphogenic protein is administered to the ligament cells. In some embodiments, the morphogenic protein is administered with a matrix. In other embodiments, the morphogenic protein is administered without a matrix.

Compositions of Ligament Cells and BMPs

The invention also provides a composition comprising ligament cells and a bone morphogenic protein. In some embodiments the ligament cells are transfected with a nucleic acid sequence encoding a morphogenic protein or a growth factor according to this invention. In some embodiments the composition further comprises a ligament cell associated matrix according to this invention.

The Bone Morphogenic Protein Family

The BMP family, named for its representative bone morphogenic/osteogenic protein family members, belongs to the TGF-β protein superfamily. Of the reported BMPs (BMP-1 to BMP-18), isolated primarily based on sequence homology, all but BMP-1 remain classified as members of the BMP family of morphogenic proteins (Ozkaynak et al., EMBO J., 9, pp. 2085-93 (1990)).

The BMP family includes other structurally-related members which are morphogenic proteins, including the drosophila decapentaplegic gene complex (DPP) products, the Vg1 product of Xenopus laevis and its murine homolog, Vgr-1 (see, e.g., Massagué, Annu. Rev. Cell Biol., 6, pp. 597-641 (1990), incorporated herein by reference).

The C-terminal domains of BMP-3, BMP-5, BMP-6, and OP-1 (BMP-7) are about 60% identical to that of BMP-2, and the C-terminal domains of BMP-6 and OP-1 are 87% identical. BMP-6 is likely the human homolog of the murine Vgr-1 (Lyons et al., Proc. Natl. Acad. Sci. U.S.A., 86, pp. 4554-59 (1989)); the two proteins are 92% identical overall at the amino acid sequence level (U.S. Pat. No. 5,459,047, incorporated herein by reference). BMP-6 is 58% identical to the Xenopus Vg-1 product.

Biochemical, Structural and Functional Properties of BMPs

The naturally occurring bone morphogens share substantial amino acid sequence homology in their C-terminal regions (domains). Typically, the above-mentioned naturally occurring osteogenic proteins are translated as a precursor, having an N-terminal signal peptide sequence typically less than about 30 residues, followed by a “pro” domain that is cleaved to yield the mature C-terminal domain of approximately 97-106 amino acids. The signal peptide is cleaved rapidly upon translation, at a cleavage site that can be predicted in a given sequence using the method of Von Heijne Nucleic Acids Research, 14, pp. 4683-4691 (1986). The pro domain typically is about three times larger than the fully processed mature C-terminal domain.

Another characteristic of the BMP protein family members is their apparent ability to dimerize. Several bone-derived OPs and BMPs are found as homo- and heterodimers in their active forms. The ability of OPs and BMPs to form heterodimers may confer additional or altered morphogenic inductive capabilities on morphogenic proteins. Heterodimers may exhibit qualitatively or quantitatively different binding affinities than homodimers for OP and BMP receptor molecules. Altered binding affinities may in turn lead to differential activation of receptors that mediate different signaling pathways, which may ultimately lead to different biological activities or outcomes. Altered binding affinities could also be manifested in a tissue or cell type-specific manner, thereby inducing only particular progenitor cell types to undergo proliferation and/or differentiation.

In some embodiments, the pair of morphogenic polypeptides have amino acid sequences each comprising a sequence that shares a defined relationship with an amino acid sequence of a reference morphogen. Herein, preferred osteogenic polypeptides share a defined relationship with a sequence present in osteogenically active human OP-1, SEQ ID NO: 1. However, any one or more of the naturally occurring or biosynthetic sequences disclosed herein similarly could be used as a reference sequence. Preferred osteogenic polypeptides share a defined relationship with at least the C-terminal six cysteine domain of human OP-1, residues 335-431 of SEQ ID NO: 1. Preferably, osteogenic polypeptides share a defined relationship with at least the C-terminal seven cysteine domain of human OP-1, residues 330-431 of SEQ ID NO: 1. That is, preferred polypeptides in a dimeric protein with bone morphogenic activity each comprise a sequence that corresponds to a reference sequence or is functionally equivalent thereto.

Functionally equivalent sequences include functionally equivalent arrangements of cysteine residues disposed within the reference sequence, including amino acid insertions or deletions which alter the linear arrangement of these cysteines, but do not materially impair their relationship in the folded structure of the dimeric morphogen protein, including their ability to form such intra- or inter-chain disulfide bonds as may be necessary for morphogenic activity. Functionally equivalent sequences further include those wherein one or more amino acid residues differs from the corresponding residue of a reference sequence, e.g., the C-terminal seven cysteine domain (also referred to herein as the conserved seven cysteine skeleton) of human OP-1, provided that this difference does not destroy bone morphogenic activity. Accordingly, conservative substitutions of corresponding amino acids in the reference sequence are preferred. Particularly preferred conservative substitutions are those fulfilling the criteria defined for an accepted point mutation in Dayhoff et al., supra, the teachings of which are incorporated by reference herein.

The osteogenic protein OP-1 has been described (see, e.g., Oppermann et al., U.S. Pat. No. 5,354,557, incorporated herein by reference). Natural-sourced osteogenic protein in its mature, native form is a glycosylated dimer typically having an apparent molecular weight of about 30-36 kDa as determined by SDS-PAGE. When reduced, the 30 kDa protein gives rise to two glycosylated peptide subunits having apparent molecular weights of about 16 kDa and 18 kDa. In the reduced state, the protein has no detectable osteogenic activity. The unglycosylated protein, which also has osteogenic activity, has an apparent molecular weight of about 27 kDa. When reduced, the 27 kDa protein gives rise to two unglycosylated polypeptides, having molecular weights of about 14 kDa to 16 kDa, capable of inducing endochondral bone formation in a mammal. Osteogenic proteins may include forms having varying glycosylation patterns, varying N-termini, and active truncated or mutated forms of native protein. As described above, particularly useful sequences include those comprising the C-terminal 96 or 102 amino acid sequences of DPP (from Drosophila), Vg1 (from Xenopus), Vgr-1 (from mouse), the OP-1 and OP-2 proteins, (see U.S. Pat. No. 5,011,691 and Oppermann et al., incorporated herein by reference), as well as the proteins referred to as BMP-2, BMP-3, BMP-4 (see WO88/00205, U.S. Pat. No. 5,013,649 and WO91/18098, incorporated herein by reference), BMP-5 and BMP-6 (see WO90/11366, PCT/US90/01630, incorporated herein by reference), BMP-8 and BMP-9.

Preferred osteogenic proteins of this invention include OP-1, OP-2, OP-3, BMP-2, BMP-3, BMP-4, BMP-5, BMP-6, BMP-8, BMP-9, BMP-10, BMP-11, BMP-15, BMP-16, DPP, Vg-1, Vgr-1, 60A protein, GDF-1, GDF-2, GDF-3, GDF-5, GDF-6, GDF-7, GDF-8, GDF-9, GDF-10, GDF-11, GDF-12, NODAL, UNIVIN, SCREW, ADMP, NEURAL, and amino acid sequence variants and homologs thereof, including species homologs, thereof. More preferred osteogenic proteins include OP-1, GDF-5, GDF-6, and GDF-7. The most preferred osteogenic protein is OP-1.

Documents disclosing these sequences, as well as their chemical and physical properties, include: OP-1 and OP-2 (U.S. Pat. No. 5,011,691; U.S. Pat. No. 5,266,683; Ozkaynak et al., EMBO J., 9, pp. 2085-2093 (1990); OP-3 (WO94/10203 (PCT US93/10520)), BMP-2, BMP-3, BMP-4, (WO88/00205; Wozney et al. Science, 242, pp. 1528-1534 (1988)), BMP-5 and BMP-6, (Celeste et al., PNAS, 87, 9843-9847 (1991)), Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (WO95/33830 (PCT/US95/07084); BMP-10 (WO94/26893 (PCT/US94/05290); BMP-11 (WO94/26892 (PCT/US94/05288); BMP-12 (WO95/16035 (PCT/US94/14030); BMP-13 (WO95/16035 (PCT/US94/14030); GDF-1 (WO92/00382 (PCT/US91/04096) and Lee et al. PNAS, 88, pp. 4250-4254 (1991); GDF-8 (WO94/21681 (PCT/US94/03019); GDF-9 (WO94/15966 (PCT/US94/00685); GDF-10 (WO95/10539 (PCT/US94/11440); GDF-11 (WO96/01845 (PCT/US95/08543); BMP-15 (WO96/36710 (PCT/US96/06540); GDF-5 (CDMP-1, MP52) (WO94/15949 (PCT/US94/00657) and WO96/14335 (PCT/US94/12814) and WO93/16099 (PCT/EP93/00350)); GDF-6 (CDMP-2, BMPl3) (WO95/01801 (PCT/US94/07762) and WO96/14335 and WO95/10635 (PCT/US94/14030)); GDF-7 (CDMP-3, BMP12) (WO95/10802 (PCT/US94/07799) and WO95/10635 (PCT/US94/14030)). The above documents are incorporated herein by reference.

In another embodiment, useful proteins include biologically active biosynthetic constructs, including novel biosynthetic morphogenic proteins and chimeric proteins designed using sequences from two or more known morphogens.

Osteogenic proteins prepared synthetically may be native, or may be non-native proteins, i.e., those not otherwise found in nature. Non-native osteogenic proteins have been synthesized using a series of consensus DNA sequences (U.S. Pat. No. 5,324,819, incorporated herein by reference). These consensus sequences were designed based on partial amino acid sequence data obtained from natural osteogenic products and on their observed homologies with other genes reported in the literature having a presumed or demonstrated developmental function.

Several of the biosynthetic consensus sequences (called consensus osteogenic proteins or “COPs”) have been expressed as fusion proteins in prokaryotes. Purified fusion proteins may be cleaved, refolded, combined with at least one MPSF (optionally in a matrix or device), implanted in an established animal model and shown to have bone- and/or cartilage-inducing activity. The currently preferred synthetic osteogenic proteins comprise two synthetic amino acid sequences designated COP-5 (SEQ. ID NO: 2) and COP-7 (SEQ. ID NO: 3).

Oppermann et al., U.S. Pat. Nos. 5,011,691 and 5,324,819, which are incorporated herein by reference, describe the amino acid sequences of COP-5 and COP-7 as shown below:

| | |
|---|---|
| COP5 | LYVDFS-DVGWDDWIVAPPGYQAFYCHGECPFPLAD |
| COP7 | LYVDFS-DVGWNDWIVAPPGYHAFYCHGECPFPLAD |
| COP5 | HFNSTN--H-AVVQTLVNSVNSKI--PKACCVPTELSA |
| COP7 | HLNSTN--H-AVVQTLVNSVNSKI--PKACCVPTELSA |
| COP5 | ISMLYLDENEKVVLKYNQEMVVEGCGCR |
| COP7 | ISMLYLDENEKVVLKYNQEMVVEGCGCR |

In these amino acid sequences, the dashes (−) are used as fillers only to line up comparable sequences in related proteins. Differences between the aligned amino acid sequences are highlighted.

The DNA and amino acid sequences of these and other BMP family members are published and may be used by those of skill in the art to determine whether a newly identified protein belongs to the BMP family. New BMP-related gene products are expected by analogy to possess at least one morphogenic activity and thus classified as a BMP.

In one preferred embodiment of this invention, the morphogenic protein comprises a pair of subunits disulfide bonded to produce a dimeric species, wherein at least one of the subunits comprises a polypeptide belonging to the BMP protein family. In another preferred embodiment of this invention, the morphogenic protein comprises a pair of subunits that produce a dimeric species formed through non-covalent interactions, wherein at least one of the subunits comprises a polypeptide belonging to the BMP protein family. Non-covalent interactions include Van der Waals, hydrogen bond, hydrophobic and electrostatic interactions. The dimeric species may be a homodimer or heterodimer and is capable of inducing cell proliferation and/or tissue formation.

In certain preferred embodiments, osteogenic proteins useful herein include those in which the amino acid sequences comprise a sequence sharing at least 70% amino acid sequence homology or “similarity”, and preferably 80% homology or similarity, with a reference morphogenic protein selected from the foregoing naturally occurring proteins. Preferably, the reference protein is human OP-1, and the reference sequence thereof is the C-terminal seven cysteine domain present in osteogenically active forms of human OP-1, residues 330-431 of SEQ ID NO: 1. In certain embodiments, a polypeptide suspected of being functionally equivalent to a reference morphogen polypeptide is aligned therewith using the method of Needleman, et al., supra, implemented conveniently by computer programs such as the Align program (DNAstar, Inc.). As noted above, internal gaps and amino acid insertions in the candidate sequence are ignored for purposes of calculating the defined relationship, conventionally expressed as a level of amino acid sequence homology or identity, between the candidate and reference sequences. In a preferred embodiment, the reference sequence is OP-1. Osteogenic proteins useful herein accordingly include allelic, phylogenetic counterpart and other variants of the preferred reference sequence, whether naturally-occurring or biosynthetically produced (e.g., including “muteins” or “mutant proteins”), as well as novel members of the general morphogenic family of proteins, including those set forth and identified above. Certain particularly preferred morphogenic polypeptides share at least 60% amino acid identity with the preferred reference sequence of human OP-1, still more preferably at least 65% amino acid identity therewith, and even more preferably, at least 70% amino acid identity therewith.

In another embodiment, useful osteogenic proteins include those sharing the conserved seven cysteine domain and sharing at least 70% amino acid sequence homology (similarity) within the C-terminal active domain, as defined herein. In still another embodiment, the osteogenic proteins of the invention can be defined as osteogenically active proteins having any one of the generic sequences defined herein, including OPX (SEQ ID NO: 4) and Generic Sequences 7 (SEQ ID NO: 5) and 8 (SEQ ID NO: 6), or Generic Sequences 9 (SEQ ID NO: 7) and 10 (SEQ ID NO: 8).

The family of bone morphogenic polypeptides useful in the present invention, and members thereof, can be defined by a generic amino acid sequence. For example, Generic Sequence 7 (SEQ ID NO: 5) and Generic Sequence 8 (SEQ ID NO: 6) are 96 and 102 amino acid sequences, respectively, and accommodate the homologies shared among preferred protein family members identified to date, including at least OP-1, OP-2, OP-3, CBMP-2A, CBMP-2B, BMP-3, 60A, DPP, Vg1, BMP-5, BMP-6, Vgr-1, and GDF-1. The amino acid sequences for these proteins are described herein and/or in the art, as summarized above. The generic sequences include both the amino acid identity shared by these sequences in the C-terminal domain, defined by the six and seven cysteine skeletons (Generic Sequences 7 and 8, respectively), as well as alternative residues for the −15 variable positions within the sequence. The generic sequences provide an appropriate cysteine skeleton where inter- or intramolecular disulfide bonds can form, and contain certain critical amino acids likely to influence the tertiary structure of the folded proteins. In addition, the generic sequences allow for an additional cysteine at position 36 (Generic Sequence 7) or position 41 (Generic Sequence 8), thereby encompassing the morphogenically active sequences of OP-2 and OP-3.

Generic Sequence 7

(SEQ ID NO: 5)

```
                        Leu Xaa Xaa Xaa Phe Xaa Xaa
                        1 5
Xaa Gly Trp Xaa Xaa Xaa Xaa Xaa Xaa Pro
                10 15
Xaa Xaa Xaa Xaa Ala Xaa Tyr Cys Xaa Gly
                20 25
Xaa Cys Xaa Xaa Pro Xaa Xaa Xaa Xaa Xaa
                30 35
Xaa Xaa Xaa Asn His Ala Xaa Xaa Xaa Xaa
                40 45
Xaa Xaa Xaa Xaa Xaa Xaa Xaa Xaa Xaa Xaa
                50 55
Xaa Xaa Xaa Cys Cys Xaa Pro Xaa Xaa Xaa
                60 65
Xaa Xaa Xaa Xaa Xaa Leu Xaa Xaa Xaa Xaa
                70 75
Xaa Xaa Xaa Val Xaa Leu Xaa Xaa Xaa Xaa
                80 85
Xaa Met Xaa Val Xaa Xaa Cys Xaa Cys Xaa
                90 95
```

wherein each Xaa independently is selected from a group of one or more specified amino acids defined as follows: “res.” means “residue” and Xaa at res.2=(Tyr or Lys); Xaa at res.3=Val or Ile); Xaa at res.4=(Ser, Asp or Glu); Xaa at res.6=(Arg, Gln, Ser, Lys or Ala); Xaa at res.7=(Asp or Glu); Xaa at res.8=(Leu, Val or Ile); Xaa at res. 11=(Gln, Leu, Asp, His, Asn or Ser); Xaa at res.12=(Asp, Arg, Asn or Glu); Xaa at res.13=(Trp or Ser); Xaa at res.14=(Ile or Val); Xaa at res.15=(Ile or Val); Xaa at res.16 (Ala or Ser); Xaa at res.18=(Glu, Gln, Leu, Lys, Pro or Arg); Xaa at res.19=(Gly or Ser); Xaa at res.20=(Tyr or Phe); Xaa at res.21=(Ala, Ser, Asp, Met, His, Gln, Leu or Gly); Xaa at res.23=(Tyr, Asn or Phe); Xaa at res.26=(Glu, His, Tyr, Asp, Gln, Ala or Ser); Xaa at res.28=(Glu, Lys, Asp, Gln or Ala); Xaa at res.30=(Ala, Ser, Pro, Gln, Ile or Asn); Xaa at res.31=(Phe, Leu or Tyr); Xaa at res.33=(Leu, Val or Met); Xaa at res.34=(Asn, Asp, Ala, Thr or Pro); Xaa at res.35=(Ser, Asp, Glu, Leu, Ala or Lys); Xaa at res.36=(Tyr, Cys, His, Ser or Ile); Xaa at res.37=(Met, Phe, Gly or Leu); Xaa at res.38=(Asn, Ser or Lys); Xaa at res.39=(Ala, Ser, Gly or Pro); Xaa at res.40=(Thr, Leu or Ser); Xaa at res.44=(Ile, Val or Thr); Xaa at res.45=(Val, Leu, Met or Ile); Xaa at res.46=(Gln or Arg); Xaa at res.47=(Thr, Ala or Ser); Xaa at res.48=(Leu or Ile); Xaa at res.49=(Val or Met); Xaa at res.50=(His, Asn or Arg); Xaa at res.51=(Phe, Leu, Asn, Ser, Ala or Val); Xaa at res.52=(Ile, Met, Asn, Ala, Val, Gly or Leu); Xaa at res.53=(Asn, Lys, Ala, Glu, Gly or Phe); Xaa at res.54=(Pro, Ser or Val); Xaa at res.55=(Glu, Asp, Asn, Gly, Val, Pro or Lys); Xaa at res.56=(Thr, Ala, Val, Lys, Asp, Tyr, Ser, Gly, Ile or His); Xaa at res.57=(Val, Ala or Ile); Xaa at res.58=(Pro or Asp); Xaa at res.59=(Lys, Leu or Glu); Xaa at res.60 (Pro, Val or Ala); Xaa at res.63=(Ala or Val); Xaa at res.65=(Thr, Ala or Glu); Xaa at res.66=(Gln, Lys, Arg or Glu); Xaa at res.67=(Leu, Met or Val); Xaa at res.68=(Asn, Ser, Asp or Gly); Xaa at res.69=(Ala, Pro or Ser); Xaa at res.70=(Ile, Thr, Val or Leu); Xaa at res.71=(Ser, Ala or Pro); Xaa at res.72=(Val, Leu, Met or Ile); Xaa at res.74=(Tyr or Phe); Xaa at res.75=(Phe, Tyr, Leu or His); Xaa at res.76=(Asp, Asn or Leu); Xaa at res.77=(Asp, Glu, Asn, Arg or Ser); Xaa at res.78=(Ser, Gln, Asn, Tyr or Asp); Xaa at res.79=(Ser, Asn, Asp, Glu or Lys); Xaa at res.80=(Asn, Thr or Lys); Xaa at res.82=(Ile, Val or Asn); Xaa at res.84=(Lys or Arg); Xaa at res.85=(Lys, Asn, Gln, His, Arg or Val); Xaa at res.86=(Tyr, Glu or His); Xaa at res.87=(Arg, Gln, Glu or Pro); Xaa at res.88=(Asn, Glu, Trp or Asp); Xaa at res.90=(Val, Thr, Ala or Ile); Xaa at res.92=(Arg, Lys, Val, Asp, Gln or Glu); Xaa at res.93=(Ala, Gly, Glu or Ser); Xaa at res.95=(Gly or Ala) and Xaa at res.97=(His or Arg).

Generic Sequence 8 (SEQ ID NO: 6) includes all of Generic Sequence 7 and in addition includes the following sequence (SEQ ID NO: 9) at its N-terminus:

| Cys Xaa Xaa Xaa Xaa | SEQ ID NO: 9 |
|---|---|
| 1 5 | |

Accordingly, beginning with residue 7, each “Xaa” in Generic Sequence 8 is a specified amino acid defined as for Generic Sequence 7, with the distinction that each residue number described for Generic Sequence 7 is shifted by five in Generic Sequence 8. Thus, “Xaa at res.2=(Tyr or Lys)” in Generic Sequence 7 refers to Xaa at res. 7 in Generic Sequence 8. In Generic Sequence 8, Xaa at res.2=(Lys, Arg, Ala or Gln); Xaa at res.3=(Lys, Arg or Met); Xaa at res.4=(His, Arg or Gln); and Xaa at res. 5=(Glu, Ser, His, Gly, Arg, Pro, Thr, or Tyr).

In another embodiment, useful osteogenic proteins include those defined by Generic Sequences 9 and 10, defined as follows.

Specifically, Generic Sequences 9 and 10 are composite amino acid sequences of the following proteins: human OP-1, human OP-2, human OP-3, human BMP-2, human BMP-3, human BMP-4, human BMP-5, human BMP-6, human BMP-8, human BMP-9, human BMP 10, human BMP-11, Drosophila 60A, Xenopus Vg-1, sea urchin UNIVIN, human CDMP-1 (mouse GDF-5), human CDMP-2 (mouse GDF-6, human BMP-13), human CDMP-3 (mouse GDF-7, human BMP-12), mouse GDF-3, human GDF-1, mouse GDF-1, chicken DORSALIN, dpp, Drosophila SCREW, mouse NODAL, mouse GDF-8, human GDF-8, mouse GDF-9, mouse GDF-10, human GDF-11, mouse GDF-11, human BMP-15, and rat BMP3b. Like Generic Sequence 7, Generic Sequence 9 is a 96 amino acid sequence that accommodates the C-terminal six cysteine skeleton and, like Generic Sequence 8, Generic Sequence 10 is a 102 amino acid sequence which accommodates the seven cysteine skeleton.

Generic Sequence 9

(SEQ ID NO: 7)

```
Xaa Xaa Xaa Xaa Xaa Xaa Xaa Xaa Xaa Xaa
1 5 10
Xaa Xaa Xaa Xaa Xaa Xaa Pro Xaa Xaa Xaa
                                15 20
Xaa Xaa Xaa Xaa Cys Xaa Gly Xaa Cys Xaa
                                25 30
Xaa Xaa Xaa Xaa Xaa Xaa Xaa Xaa Xaa Xaa
                                35 40
Xaa Xaa Xaa Xaa Xaa Xaa Xaa Xaa Xaa Xaa
                                45 50
Xaa Xaa Xaa Xaa Xaa Xaa Xaa Xaa Xaa Xaa
                                55 60
Xaa Cys Xaa Pro Xaa Xaa Xaa Xaa Xaa Xaa
                                65 70
Xaa Xaa Leu Xaa Xaa Xaa Xaa Xaa Xaa Xaa
                                75 80
Xaa Xaa Xaa Xaa Xaa Xaa Xaa Xaa Xaa Xaa
                                85 90
Xaa Xaa Xaa Cys Xaa Cys Xaa
                                95
```

wherein each Xaa is independently selected from a group of one or more specified amino acids defined as follows: “res.” means “residue” and Xaa at res. 1=(Phe, Leu or Glu); Xaa at res. 2=(Tyr, Phe, His, Arg, Thr, Lys, Gln, Val or Glu); Xaa at res. 3=(Val, Ile, Leu or Asp); Xaa at res. 5=(Ser, Asp, Glu, Asn or Phe); Xaa at res. 5=(Phe or Glu); Xaa at res. 6=(Arg, Gln, Lys, Ser, Glu, Ala or Asn); Xaa at res. 7=(Asp, Glu, Leu, Ala or Gln); Xaa at res. 8=(Leu, Val, Met, Ile or Phe); Xaa at res. 9=(Gly, His or Lys); Xaa at res. 10=(Trp or Met); Xaa at res. 11=(Gln, Leu, His, Glu, Asn, Asp, Ser or Gly); Xaa at res. 12=(Asp, Asn, Ser, Lys, Arg, Glu or His); Xaa at res. 13=(Trp or Ser); Xaa at res. 14=(Ile or Val); Xaa at res. 15=(Ile or Val); Xaa at res. 16=(Ala, Ser, Tyr or Trp); Xaa at res. 18=(Glu, Lys, Gln, Met, Pro, Leu, Arg, His or Lys); Xaa at res. 19=(Gly, Glu, Asp, Lys, Ser, Gln, Arg or Phe); Xaa at res. 20=(Tyr or Phe); Xaa at res. 21=(Ala, Ser, Gly, Met, Gln, His, Glu, Asp, Leu, Asn, Lys or Thr); Xaa at res. 22=(Ala or Pro); Xaa at res. 23=(Tyr, Phe, Asn, Ala or Arg); Xaa at res. 24=(Tyr, His, Glu, Phe or Arg); Xaa at res. 26=(Glu, Asp, Ala, Ser, Tyr, His, Lys, Arg, Gln or Gly); Xaa at res. 28=(Glu, Asp, Leu, Val, Lys, Gly, Thr, Ala or Gln); Xaa at res. 30=(Ala, Ser, Ile, Asn, Pro, Glu, Asp, Phe, Gln or Leu); Xaa at res. 31=(Phe, Tyr, Leu, Asn, Gly or Arg); Xaa at res. 0.32=(Pro, Ser, Ala or Val); Xaa at res. 33=(Leu, Met, Glu, Phe or Val); Xaa at res. 34=(Asn, Asp, Thr, Gly, Ala, Arg, Leu or Pro); Xaa at res. 35=(Ser, Ala, Glu, Asp, Thr, Leu, Lys, Gln or His); Xaa at res. 36=(Tyr, His, Cys, Ile, Arg, Asp, Asn, Lys, Ser, Glu or Gly); Xaa at res. 37=(Met, Leu, Phe, Val, Gly or Tyr); Xaa at res. 38=(Asn, Glu, Thr, Pro, Lys, His, Gly, Met, Val or Arg); Xaa at res. 39=(Ala, Ser, Gly, Pro or Phe); Xaa at res. 40=(Thr, Ser, Leu, Pro, His or Met); Xaa at res. 41=(Asn, Lys, Val, Thr or Gln); Xaa at res. 42=(His, Tyr or Lys); Xaa at res. 43=(Ala, Thr, Leu or Tyr); Xaa at res. 44=(Ile, Thr, Val, Phe, Tyr, Met or Pro); Xaa at res. 45=(Val, Leu, Met, Ile or His); Xaa at res. 46=(Gln, Arg or Thr); Xaa at res. 47=(Thr, Ser, Ala, Asn or His); Xaa at res. 48=(Leu, Asn or Ile); Xaa at res. 49=(Val, Met, Leu, Pro or Ile); Xaa at res. 50 (His, Asn, Arg, Lys, Tyr or Gln); Xaa at res. 51=(Phe, Leu, Ser, Asn, Met, Ala, Arg, Glu, Gly or Gln); Xaa at res. 52=(Ile, Met, Leu, Val, Lys, Gln, Ala or Tyr); Xaa at res. 53=(Asn, Phe, Lys, Glu, Asp, Ala, Gln, Gly, Leu or Val); Xaa at res. 54=(Pro, Asn, Ser, Val or Asp); Xaa at res. 55=(Glu, Asp, Asn, Lys, Arg, Ser, Gly, Thr, Gln, Pro or His); Xaa at res. 56=(Thr, His, Tyr, Ala, Ile, Lys, Asp, Ser, Gly or Arg); Xaa at res. 57=(Val, Ile, Thr, Ala, Leu or Ser); Xaa at res. 58=(Pro, Gly, Ser, Asp or Ala); Xaa at res. 59=(Lys, Leu, Pro, Ala, Ser, Glu, Arg or Gly); Xaa at res. 60=(Pro, Ala, Val, Thr or Ser); Xaa at res. 61=(Cys, Val or Ser); Xaa at res. 63=(Ala, Val or Thr); Xaa at res. 65=(Thr, Ala, Glu, Val, Gly, Asp or Tyr); Xaa at res. 66=(Gln, Lys, Glu, Arg or Val); Xaa at res. 67=(Leu, Met, Thr or Tyr); Xaa at res. 68=(Asn, Ser, Gly, Thr, Asp, Glu, Lys or Val); Xaa at res. 69=(Ala, Pro, Gly or Ser); Xaa at res. 70=(Ile, Thr, Leu or Val); Xaa at res. 71=(Ser, Pro, Ala, Thr, Asn or Gly); Xaa at res. 2=(Val, Ile, Leu or Met); Xaa at res. 74=(Tyr, Phe, Arg, Thr, Tyr or Met); Xaa at res. 75=(Phe, Tyr, His, Leu, Ile, Lys, Gln or Val); Xaa at res. 76=(Asp, Leu, Asn or Glu); Xaa at res. 77=(Asp, Ser, Arg, Asn, Glu, Ala, Lys, Gly or Pro); Xaa at res. 78=(Ser, Asn, Asp, Tyr, Ala, Gly, Gln, Met, Glu, Asn or Lys); Xaa at res. 79=(Ser, Asn, Glu, Asp, Val, Lys, Gly, Gln or Arg); Xaa at res. 80=(Asn, Lys, Thr, Pro, Val, Ile, Arg, Ser or Gln); Xaa at res. 81=(Val, Ile, Thr or Ala); Xaa at res. 82=(Ile, Asn, Val, Leu, Tyr, Asp or Ala); Xaa at res. 83=(Leu, Tyr, Lys or Ile); Xaa at res. 84=(Lys, Arg, Asn, Tyr, Phe, Thr, Glu or Gly); Xaa at res. 85=(Lys, Arg, His, Gln, Asn, Glu or Val); Xaa at res. 86 (Tyr, His, Glu or Ile); Xaa at res. 87=(Arg, Glu, Gln, Pro or Lys); Xaa at res. 88=(Asn, Asp, Ala, Glu, Gly or Lys); Xaa at res. 89=(Met or Ala); Xaa at res. 90=(Val, Ile, Ala, Thr, Ser or Lys); Xaa at res 91=(Val or Ala); Xaa at res. 92=(Arg, Lys, Gln, Asp, Glu, Val, Ala, Ser or Thr); Xaa at res. 93=(Ala, Ser, Glu, Gly, Arg or Thr); Xaa at res. 95=(Gly, Ala or Thr); Xaa at res. 97=(His, Arg, Gly, Leu or Ser). Further, after res. 53 in rBMP3b and mGDF-10 there is an Ile; after res. 54 in GDF-1 there is a T; after res. 54 in BMP3 there is a V; after res. 78 in BMP-8 and Dorsalin there is a G; after res. 37 in hGDF-1 there is Pro, Gly, Gly, Pro.

Generic Sequence 10 (SEQ ID NO: 8) includes all of Generic Sequence 9 (SEQ ID NO: 7) and in addition includes the following sequence (SEQ ID NO: 9) at its N-terminus:

| Cys Xaa Xaa Xaa Xaa | SEQ ID NO: 9 |
|---|---|
| 1 5 | |

Accordingly, beginning with residue 6, each “Xaa” in Generic Sequence 10 is a specified amino acid defined as for Generic Sequence 9, with the distinction that each residue number described for Generic Sequence 9 is shifted by five in Generic Sequence 10. Thus, “Xaa at res. 1=(Tyr, Phe, His, Arg, Thr, Lys, Gln, Val or Glu)” in Generic Sequence 9 refers to Xaa at res. 6 in Generic Sequence 10. In Generic Sequence 10, Xaa at res. 2=(Lys, Arg, Gln, Ser, His, Glu, Ala, or Cys); Xaa at res. 3=(Lys, Arg, Met, Lys, Thr, Leu, Tyr, or Ala); Xaa at res. 4=(His, Gln, Arg, Lys, Thr, Leu, Val, Pro, or Tyr); and Xaa at res. 5=(Gln, Thr, His, Arg, Pro, Ser, Ala, Gln, Asn, Tyr, Lys, Asp, or Leu).

As noted above, certain currently preferred bone morphogenic polypeptide sequences useful in this invention have greater than 60% identity, preferably greater than 65% identity, more preferably greater than 70% identity, with the amino acid sequence defining the preferred reference sequence of hOP-1. These particularly preferred sequences include allelic and phylogenetic counterpart variants of the OP-1 and OP-2 proteins, including the Drosophila 60A protein. Accordingly, in certain particularly preferred embodiments, useful morphogenic proteins include active proteins comprising pairs of polypeptide chains within the generic amino acid sequence herein referred to as “OPX” (SEQ ID NO: 4), which defines the seven cysteine skeleton and accommodates the homologies between several identified variants of OP-1 and OP-2. As described therein, each Xaa at a given position independently is selected from the residues occurring at the corresponding position in the C-terminal sequence of mouse or human OP-1 or OP-2.

```
Cys Xaa Xaa His Glu Leu Tyr Val Ser Phe Xaa Asp
    1 5 10
Leu Gly Trp Xaa Asp Trp Xaa Ile Ala Pro Xaa Gly
                  15 20
Tyr Xaa Ala Tyr Tyr Cys Glu Gly Glu Cys Xaa Phe
  25 30 35
Pro Leu Xaa Ser Xaa Met Asn Ala Thr Asn His Ala
                          40 45
Ile Xaa Gln Xaa Leu Val His Xaa Xaa Xaa Pro Xaa
          50 55 60
Xaa Val Pro Lys Xaa Cys Cys Ala Pro Thr Xaa Leu
                                  65 70
Xaa Ala Xaa Ser Val Leu Tyr Xaa Asp Xaa Ser Xaa
                  75 80
Asn Val Ile Leu Xaa Lys Xaa Arg Asn Met Val Val
  85 90 95
Xaa Ala Cys Gly Cys His
                        100
```

wherein Xaa at res. 2=(Lys or Arg); Xaa at res. 3=(Lys or Arg); Xaa at res. 11=(Arg or Gln); Xaa at res. 16=(Gln or Leu); Xaa at res. 19=(Ile or Val); Xaa at res. 23=(Glu or Gln); Xaa at res. 26=(Ala or Ser); Xaa at res. 35=(Ala or Ser); Xaa at res. 39 (Asn or Asp); Xaa at res. 41=(Tyr or Cys); Xaa at res. 50=(Val or Leu); Xaa at res. 52=(Ser or Thr); Xaa at res. 56=(Phe or Leu); Xaa at res. 57=(Ile or Met); Xaa at res. 58=(Asn or Lys); Xaa at res. 60=(Glu, Asp or Asn); Xaa at res. 61=(Thr, Ala or Val); Xaa at res. 65=(Pro or Ala); Xaa at res. 71=(Gln or Lys); Xaa at res. 73=(Asn or Ser); Xaa at res. 75=(Ile or Thr); Xaa at res. 80=(Phe or Tyr); Xaa at res. 82=(Asp or Ser); Xaa at res. 84=(Ser or Asn); Xaa at res. 89=(Lys or Arg); Xaa at res. 91=(Tyr or His); and Xaa at res. 97=(Arg or Lys).

In still another preferred embodiment, useful osteogenically active proteins have polypeptide chains with amino acid sequences comprising a sequence encoded by a nucleic acid that hybridizes, under low, medium or high stringency hybridization conditions, to DNA or RNA encoding reference morphogen sequences, e.g., C-terminal sequences defining the conserved seven cysteine domains of OP-1, OP-2, BMP-2, BMP-4, BMP-5, BMP-6, 60A, GDF-3, GDF-6, GDF-7 and the like. As used herein, high stringent hybridization conditions are defined as hybridization according to known techniques in 40% formamide, 5×SSPE, 5× Denhardt's Solution, and 0.1% SDS at 37° C. overnight, and washing in 0.1×SSPE, 0.1% SDS at 50° C. Standard stringent conditions are well characterized in commercially available, standard molecular cloning texts. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984): Nucleic Acid Hybridization (B. D. Hames & S. J. Higgins eds. 1984); and B. Perbal, A Practical Guide To Molecular Cloning (1984), the disclosures of which are incorporated herein by reference.

As noted above, proteins useful in the present invention generally are dimeric proteins comprising a folded pair of the above polypeptides. Such morphogenic proteins are inactive when reduced, but are active as oxidized homodimers and when oxidized in combination with others of this invention to produce heterodimers. Thus, members of a folded pair of morphogenic polypeptides in a morphogenically active protein can be selected independently from any of the specific polypeptides mentioned above.

The bone morphogenic proteins useful in the materials and methods of this invention include proteins comprising any of the polypeptide chains described above, whether isolated from naturally-occurring sources, or produced by recombinant DNA or other synthetic techniques, and includes allelic and phylogenetic counterpart variants of these proteins, as well as muteins thereof, and various truncated and fusion constructs. Deletion or addition mutants also are envisioned to be active, including those which may alter the conserved C-terminal six or seven cysteine domain, provided that the alteration does not functionally disrupt the relationship of these cysteines in the folded structure. Accordingly, such active forms are considered the equivalent of the specifically described constructs disclosed herein. The proteins may include forms having varying glycosylation patterns, varying N-termini, a family of related proteins having regions of amino acid sequence homology, and active truncated or mutated forms of native or biosynthetic proteins, produced by expression of recombinant DNA in host cells.

The bone morphogenic proteins contemplated herein can be expressed from intact or truncated cDNA or from synthetic DNAs in prokaryotic or eukaryotic host cells, and purified, cleaved, refolded, and dimerized to form morphogenically active compositions. Currently preferred host cells include, without limitation, prokaryotes including E. coli or eukaryotes including yeast, or mammalian cells, such as CHO, COS or BSC cells. One of ordinary skill in the art will appreciate that other host cells can be used to advantage. Detailed descriptions of the bone morphogenic proteins useful in the practice of this invention, including how to make, use and test them for osteogenic activity, are disclosed in numerous publications, including U.S. Pat. Nos. 5,266,683 and 5,011,691, the disclosures of which are incorporated by reference herein, as well as in any of the publications recited herein, the disclosures of which are incorporated herein by reference.

Thus, in view of this disclosure and the knowledge available in the art, skilled genetic engineers can isolate genes from cDNA or genomic libraries of various different biological species, which encode appropriate amino acid sequences, or construct DNAs from oligonucleotides, and then can express them in various types of host cells, including both prokaryotes and eukaryotes, to produce large quantities of active proteins capable of stimulating endochondral bone morphogenesis in a mammal.

Pharmaceutical Compositions

The pharmaceutical compositions provided by this invention comprise at least one and optionally more than one morphogenic protein combinations that are capable of inducing tissue formation when administered or implanted into a patient. The compositions of this invention will be administered at an effective dose to induce formation of ligament tissue at the treatment site selected according to the particular clinical condition addressed. Determination of a preferred pharmaceutical formulation and a therapeutically efficient dose regiment for a given application is well within the skill of the art taking into consideration, for example, the administration mode, the condition and weight of the patient, the extent of desired treatment and the tolerance of the patient for the treatment.

Doses expected to be suitable starting points for optimizing treatment regiments are based on the results of in vitro assays, and ex vivo or in vivo assays. Based on the results of such assays, a range of suitable morphogenic protein and/or growth factor concentrations can be selected to test at a treatment site in animals and then in humans.

Administration of the morphogenic proteins, including isolated and purified forms of morphogenic protein complexes, their salts or pharmaceutically acceptable derivatives thereof, may be accomplished using any of the conventionally accepted modes of administration of agents which exhibit immunosuppressive activity.

The pharmaceutical compositions comprising a morphogenic protein may be in a variety of forms. These include, for example, solid, semi-solid and liquid dosage forms such as tablets, pills, powders, liquid solutions or suspensions, suppositories, and injectable and infusible solutions. The preferred form depends on the intended mode of administration and therapeutic application and may be selected by one skilled in the art. Modes of administration may include oral, parenteral, subcutaneous, intravenous, intralesional or topical administration. In most cases, the pharmaceutical compositions will be administered in the vicinity of the treatment site in need of ligament regeneration or repair.

The pharmaceutical compositions comprising a morphogenic protein may, for example, be placed into sterile, isotonic formulations with or without cofactors which stimulate uptake or stability. The formulation is preferably liquid, or may be lyophilized powder. For example, the morphogenic protein may be diluted with a formulation buffer comprising 5.0 mg/ml citric acid monohydrate, 2.7 mg/ml trisodium citrate, 41 mg/ml mannitol, 1 mg/ml glycine and 1 mg/ml polysorbate 20. This solution can be lyophilized, stored under refrigeration and reconstituted prior to administration with sterile Water-For-Injection (USP).

The compositions also will preferably include conventional pharmaceutically acceptable carriers well known in the art (see, e.g., Remington's Pharmaceutical Sciences, 16th Ed., Mac Publishing Company (1980)). Such pharmaceutically acceptable carriers may include other medicinal agents, carriers, genetic carriers, adjuvants, excipients, etc., such as human serum albumin or plasma preparations. The compositions are preferably in the form of a unit dose and will usually be administered as a dose regiment that depends on the particular tissue treatment.

The pharmaceutical compositions may also be administered using, for example, microspheres, liposomes, other microparticulate delivery systems or sustained release formulations placed in, near, or otherwise in communication with affected tissues or the bloodstream bathing those tissues.

Liposomes containing a morphogenic protein can be prepared by well-known methods (See, e.g. DE 3,218,121; Epstein et al., Proc. Natl. Acad. Sci. U.S.A., 82, pp. 3688-92 (1985); Hwang et al., Proc. Natl. Acad. Sci. U.S.A., 77, pp. 4030-34 (1980); U.S. Pat. Nos. 4,485,045 and 4,544,545). Ordinarily the liposomes are of the small (about 200-800 Angstroms) unilamellar type in which the lipid content is greater than about 30 mol. % cholesterol. The proportion of cholesterol is selected to control the optimal rate of morphogenic protein release.

The morphogenic proteins may also be attached to liposomes containing other biologically active molecules such as immunosuppressive agents, cytokines, etc., to modulate the rate and characteristics of tissue induction. Attachment of morphogenic proteins and/or growth factors to liposomes may be accomplished by any known cross-linking agent such as heterobifunctional cross-linking agents that have been widely used to couple toxins or chemotherapeutic agents to antibodies for targeted delivery. Conjugation to liposomes can also be accomplished using the carbohydrate-directed cross-linking reagent 4-(4-maleimidophenyl) butyric acid hydrazide (MPBH) (Duzgunes et al., J. Cell. Biochem. Abst. Suppl. 16E 77 (1992)).

Carriers

The morphogenic proteins may be dispersed in an implantable biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and ethyl-L-glutamate (Sidman et al., Biopolymers, 22, pp. 547-56 (1985)); poly(2-hydroxyethyl-methacrylate) or ethylene vinyl acetate (Langer et al., J. Biomed. Mater. Res., 15, pp. 167-277 (1981); Langer, Chem. Tech., 12, pp. 98-105 (1982)).

In one embodiment of this invention, the carrier comprises a biocompatible matrix made up of particles or porous materials. The pores are preferably of a dimension to permit progenitor cell migration and subsequent differentiation and proliferation. Various matrices known in the art can be employed (see, e.g., U.S. Pat. Nos. 4,975,526; 5,162,114; 5,171,574 and WO 91/18558, which are herein incorporated by reference).

The particle size should be within the range of 70 μm-850 μm, preferably 70 μm-420 μm, most preferably 150 μm-420 μm. The matrix may be fabricated by close packing particulate material into a shape spanning the particular tissue defect to be treated. Alternatively, a material that is biocompatible, and preferably biodegradable in vivo may be structured to serve as a temporary scaffold and substratum for recruitment of migratory progenitor cells, and as a base for their subsequent anchoring and proliferation.

Useful matrix materials comprise, for example, collagen; homopolymers or copolymers of glycolic acid, lactic acid, and butyric acid, including derivatives thereof; and ceramics, such as hydroxyapatite, tricalcium phosphate and other calcium phosphates. Various combinations of these or other suitable matrix materials also may be useful as determined by the assays set forth herein.

Currently preferred carriers include particulate, demineralized, guanidine-extracted, species-specific (allogenic) bone, and specially treated particulate, protein-extracted, demineralized xenogenic bone. Optionally, such xenogenic bone powder matrices also may be treated with proteases such as trypsin. Preferably, the xenogenic matrices are treated with one or more fibril modifying agents to increase the intraparticle intrusion volume (porosity) and surface area. Useful modifying agents include solvents such as dichloromethane, trichloroacetic acid, acetonitrile and acids such as trifluoroacetic acid and hydrogen fluoride. The currently preferred fibril-modifying agent useful in formulating the matrices of this invention is a heated aqueous medium, preferably an acidic aqueous medium having a pH less than about pH 4.5, most preferably having a pH within the range of about pH 2-pH 4. A currently preferred heated acidic aqueous medium is 0.1% acetic acid which has a pH of about 3. Heating demineralized, delipidated, guanidine-extracted bone collagen in an aqueous medium at elevated temperatures (e.g., in the range of about 37° C.-65° C., preferably in the range of about 45° C.-60° C.) for approximately one hour generally is sufficient to achieve the desired surface morphology. Although the mechanism is not clear, it is hypothesized that the heat treatment alters the collagen fibrils, resulting in an increase in the particle surface area.

Demineralized guanidine-extracted xenogenic bovine bone comprises a mixture of additional materials that may be fractionated further using standard biomolecular purification techniques. For example, chromatographic separation of extract components followed by addition back to active matrix of the various extract fractions corresponding to the chromatogram peaks may be used to improve matrix properties by fractionating away inhibitors of bone or tissue-inductive activity.

The matrix may also be substantially depleted in residual heavy metals. Treated as disclosed herein, individual heavy metal concentrations in the matrix can be reduced to less than about 1 ppm.

One skilled in the art may create a biocompatible matrix of choice having a desired porosity or surface microtexture useful in the production of morphogenic protein compositions to promote bone or other tissue induction, or as a biodegradable sustained release implant. In addition, synthetically formulated matrices, prepared as disclosed herein, may be used.

General Consideration of Matrix Properties

In some embodiments, the carrier may be a biodegradable-synthetic or a synthetic-inorganic matrix (e.g., hydroxyapatite (HAP), collagen, carboxymethyl-cellulose, tricalcium phosphate, polylactic acid, polyglycolic acid, polybutyric acid and various copolymers thereof.)

Matrix geometry, particle size, the presence of surface charge, and the degree of both intra- and inter-particle porosity are all important to successful matrix performance. Studies have shown that surface charge, particle size, the presence of mineral, and the methodology for combining matrix and morphogenic proteins all play a role in achieving successful tissue induction.

The sequential cellular reactions in the interface of the matrix/osteogenic protein implants are complex. The multistep cascade includes: binding of fibrin and fibronectin to implanted matrix, migration and proliferation of mesenchymal cells, differentiation of the progenitor cells and ligament formation.

A successful carrier for morphogenic protein should perform several important functions. It should act as a slow release delivery system of morphogenic protein, protect the morphogenic protein from non-specific proteolysis, and should accommodate each step of the cellular responses involved in progenitor cell induction during tissue development.

In addition, selected materials must be biocompatible in vivo and preferably biodegradable; the carrier preferably acts as a temporary scaffold until replaced completely by new bone or tissue. Polylactic acid (PLA), polyglycolic acid (PGA), and various combinations have different dissolution rates in vivo.

The matrix material prepared from xenogenic bone and treated as disclosed herein, produces an implantable material useful in a variety of clinical settings. In addition to its use as a matrix for bone formation in various orthopedic, periodontal, and reconstructive procedures, the matrix also may be used as a sustained release carrier, or as a collagenous coating for orthopedic or general prosthetic implants.

The matrix may be shaped as desired in anticipation of surgery or shaped by the physician or technician during surgery. It is preferred to shape the matrix to span a tissue defect and to take the desired form of the new tissue. Thus, the material may be used for topical, subcutaneous, intraperitoneal, or intramuscular implants. In ligament formation procedures, the material is slowly absorbed by the body and is replaced by ligament in the shape of or very nearly the shape of the implant.

The matrix may comprise a shape-retaining solid made of loosely-adhered particulate material, e.g., collagen. It may also comprise a molded, porous solid, or simply an aggregation of close-packed particles held in place by surrounding tissue. Masticated muscle or other tissue may also be used. The matrix may also take the form of a paste or a hydrogel.

When the carrier material comprises a hydrogel matrix, it refers to a three dimensional network of cross-linked hydrophilic polymers in the form of a gel substantially composed of water, preferably but not limited to gels being greater than 90% water. Hydrogel matrices can carry a net positive or net negative charge, or may be neutral. A typical net negative charged matrix is alginate. Hydrogels carrying a net positive charge may be typified by extracellular matrix components such as collagen and laminin. Examples of commercially available extracellular matrix components include Matrigel™ and Vitrogen™. An example of a net neutral hydrogel is highly crosslinked polyethylene oxide, or polyvinyalcohol.

Various growth factors, cytokines, hormones, trophic agents and therapeutic compositions including antibiotics and chemotherapeutic agents, enzymes, enzyme inhibitors and other bioactive agents also may be adsorbed onto or dispersed within the carrier material comprising the morphogenic protein, and will also be released over time at the implantation site as the matrix material is slowly absorbed.

Other Tissue-Specific Matrices

In addition to the naturally-derived bone matrices described above, useful matrices may also be formulated synthetically by adding together reagents that have been appropriately modified. One example of such a matrix is the porous, biocompatible, in vivo biodegradable synthetic matrix disclosed in WO91/18558, the disclosure of which is hereby incorporated by reference.

Briefly, the matrix comprises a porous crosslinked structural polymer of biocompatible, biodegradable collagen, most preferably tissue-specific collagen, and appropriate, tissue-specific glycosaminoglycans as tissue-specific cell attachment factors. Bone tissue-specific collagen (e.g., Type I collagen) derived from a number of sources may be suitable for use in these synthetic matrices, including soluble collagen, acid-soluble collagen, collagen soluble in neutral or basic aqueous solutions, as well as those collagens which are commercially available. In addition, Type II collagen, as found in cartilage, also may be used in combination with Type I collagen.

Glycosaminoglycans (GAGs) or mucopolysaccharides are polysaccharides made up of residues of hexoamines glycosidically bound and alternating in a more-or-less regular manner with either hexouronic acid or hexose moieties. GAGs are of animal origin and have a tissue specific distribution (see, e.g., Dodgson et al., in Carbohydrate Metabolism and its Disorders, Dickens et al., eds., Vol. 1, Academic Press (1968)). Reaction with the GAGs also provides collagen with another valuable property, i.e., inability to provoke an immune reaction (foreign body reaction) from an animal host.

Useful GAGs include those containing sulfate groups, such as hyaluronic acid, heparin, heparin sulfate, chondroitin 6-sulfate, chondroitin 4-sulfate, dermatan sulfate, and keratin sulfate. For osteogenic devices, chondroitin 6-sulfate currently is preferred. Other GAGs also may be suitable for forming the matrix described herein, and those skilled in the art will either know or be able to ascertain other suitable GAGs using no more than routine experimentation. For a more detailed description of mucopolysaccharides, see Aspinall, Polysaccharides, Pergamon Press, Oxford (1970).

Collagen can be reacted with a GAG in aqueous acidic solutions, preferably in diluted acetic acid solutions. By adding the GAG dropwise into the aqueous collagen dispersion, coprecipitates of tangled collagen fibrils coated with GAG results. This tangled mass of fibers then can be homogenized to form a homogeneous dispersion of fine fibers and then filtered and dried.

Insolubility of the collagen-GAG products can be raised to the desired degree by covalently cross-linking these materials, which also serves to raise the resistance to resorption of these materials. In general, any covalent G60 cross-linking method suitable for cross-linking collagen also is suitable for cross-linking these composite materials, although cross-linking by a dehydrothermal process is preferred.

When dry, the cross-linked particles are essentially spherical with diameters of about 500 μm. Scanning electron microscopy shows pores of about 20 μm on the surface and 40 μm on the interior. The interior is made up of both fibrous and sheet-like structures, providing surfaces for cell attachment. The voids interconnect, providing access to the cells throughout the interior of the particle. The material appears to be roughly 99.5% void volume, making the material very efficient in terms of the potential cell mass that can be grown per gram of microcarrier.

Another useful synthetic matrix is one formulated from biocompatible, in vivo biodegradable synthetic polymers, such as those composed of glycolic acid, lactic acid and/or butyric acid, including copolymers and derivatives thereof. These polymers are well described in the art and are available commercially. For example, polymers composed of polylactic acid (e.g., MW 100 ka), 80% polylactide/20% glycoside or poly 3-hydroxybutyric acid (e.g., MW 30 ka) all may be purchased from PolySciences, Inc. The polymer compositions generally are obtained in particulate form and the morphogenic devices preferably fabricated under nonaqueous conditions (e.g., in an ethanol-trifluoroacetic acid solution, EtOH/TFA) to avoid hydrolysis of the polymers. In addition, one can alter the morphology of the particulate polymer compositions, for example to increase porosity, using any of a number of particular solvent treatments known in the art.

The naturally-sourced, synthetic and recombinant morphogenic proteins as set forth above, as well as other constructs, can be combined and dispersed in a suitable matrix preparation using any of the methods described. In general, about 500-1000 ng of active morphogenic protein are combined with 25 mg of the inactive carrier matrix for rat bioassays. In larger animals, typically about 0.8-1 mg of active morphogenic protein per gram of carrier is used. The optimal ratios of morphogenic protein to carrier for a specific combination may be determined empirically by those of skill in the art according to the procedures set forth herein. Greater amounts may be used for large implants.

### EXAMPLES

### Example 1

### Cell Proliferation in Control and OP-1-Treated Rat MCL Cells

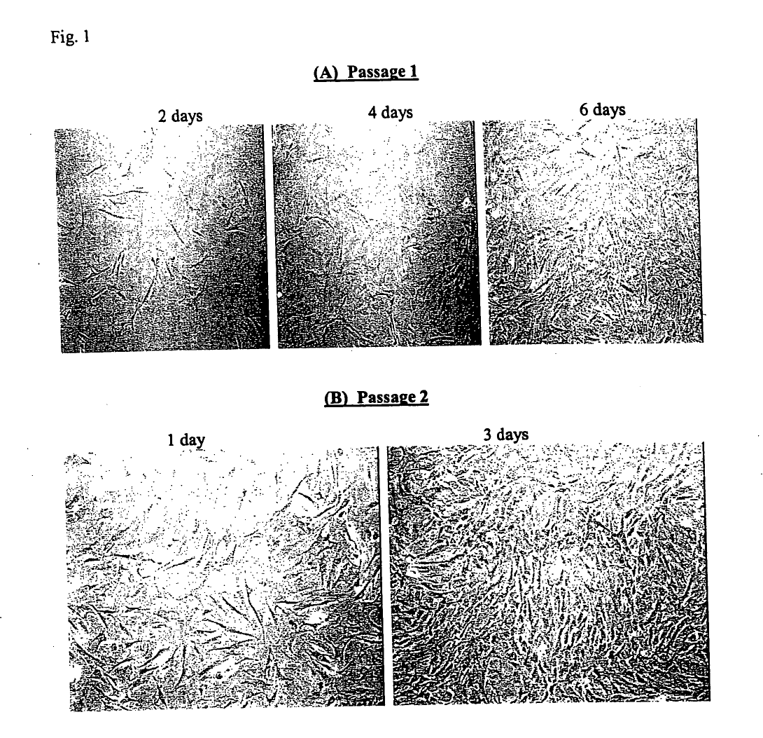

MCLs of Long Evans rats were surgically excised from surrounding connective tissue at the knee joints, rinsed with HBSS, cut into small pieces and cultured in DMEM/F12 (1:1) medium with 10% FBS supplemented with 30 μg/ml of gentamicin at 37° C. with 5% CO₂. Cells began to emerge from the tissue pieces and attach to the surface of the culture dishes at 3-4 days in culture. After 6-7 days the tissue pieces were removed and the attached cells were cultured in fresh media until confluent. The cells were then subcultured until confluent and frozen in liquid N₂. FIGS. 1A and 1B show the morphology of the control cells as a function of time. The cells that diffused out of the ligament pieces and became attached to the tissue culture dish exhibited the characteristic elongated shape and spindle-shaped nuclei and their gross morphology from passage 1 and 2 were similar.

For experimentation, cells were revived from the frozen stock in 100 mm or 150 mm dishes until confluent and subcultured at a cell density of 4×10⁴ cells/ml.

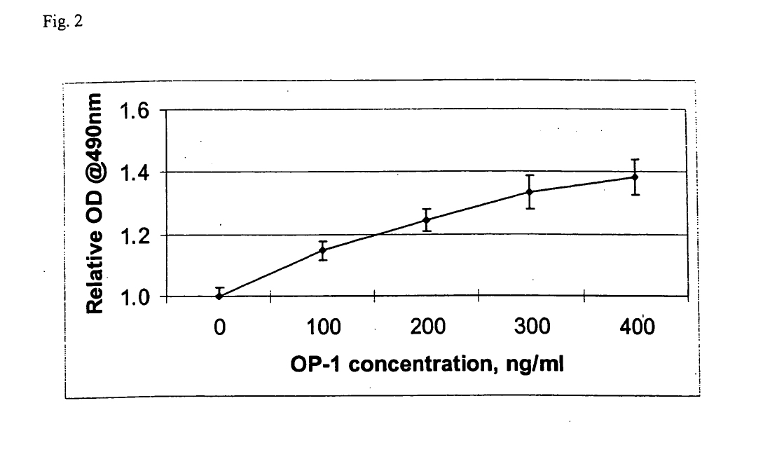

Cell proliferation was evaluated by a tetrazolium calorimetric assay (CellTiter96AQ Cell Proliferation Assay. Promega, Madison, Wis.). Briefly, cells were cultured in 96-well plates until confluent and subsequently treated with 0, 100, 200, 300, and 400 ng/ml of OP-1 in serum-free DMEM/F12 (1:1) for 24 hours. After the media were removed, the cultures were rinsed with sterile PBS. 100 μl of media containing 1% of BSA plus 20 μl of 96AQ reagent were added to each well and incubated at 37° C. for 4 hours to permit color development. The developed color was measured at 490 nm using a MRX microplate reader (Dynex Technologies, Chantilly Va.). Treatment of MCL cells with varying concentrations of OP-1 in serum free media resulted in a dose-dependent increase of cell proliferation, reaching about 40% increase for cells treated with 400 ng/ml of OP-1 (see FIG. 2).

### Example 2

### Alkaline Phosphatase (AP) Activity in Control and OP-1-Treated Rat MCL Cells

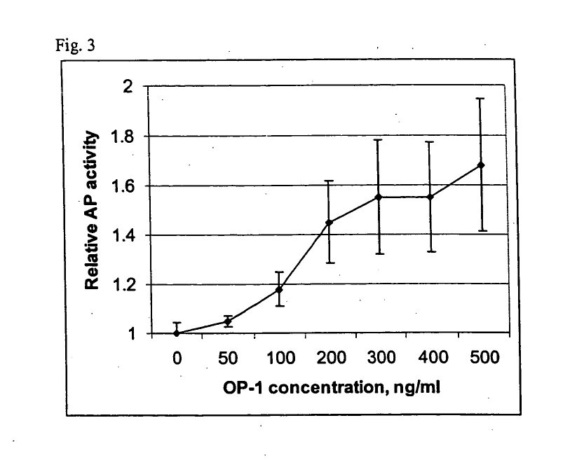

MCLs of young adult male rats were excised and the cells were cultured for experimentation as in Example 1. Confluent cells grown in 48-well plates were treated in serum-free DMEM/F12 (1:1) medium for 48 hours with 0, 50, 100, 200, 300, 400, and 500 ng/ml of OP-1. Control cells were treated with an equal amount of solvent vehicle. The cells were lysed by sonication in 0.1% Triton X-100 in PBS (100 μl/well) for 5 minutes at room temperature. The total cellular alkaline phosphatase (AP) activity was measured using a commercial assay kit (Sigma Chemical Co.) as described in Yeh et al., Endocrinology 137: 1921-31 (1996). Reactions were terminated by the addition of 0.5N NaOH. Absorbance of the reaction mixture was measured at 405 nm using a MRX microplate reader. Protein was measured according to the method described in Bradford, Anal. Biochem. 72: 248-54 (1976) using BSA as a standard. AP activity was expressed as nanomoles of p-nitrophenol liberated per μg of total cellular protein. OP-1 increased AP activity in primary cultures of rat MCL cells in a dose-dependant manner, reaching about 70% increase for cells treated with 500 ng/ml of OP-1 as compared to control untreated cells (see FIG. 3).

### Example 3

### Expression of Six1, Scleraxis, Run2x/Cbfa, Type I Collagen, and BMP Receptors in Control and OP-1-Treated Rat MCL Cells

Messenger RNA expression levels of Six1, scleraxis, Runx2/Cbfa1, type I collagen and BMP receptors ActR-I, BMPR-IA, BMPR-IB, and BMPR-II was measured in control and OP-1-treated cells by Northern blot analysis. Six1, a novel murine homeobox-containing gene, has been suggested as a specific molecular marker for limb tendons and ligaments (Oliver et al., Development 121: 793-805 (1995)). Scleraxis, a helix-loop-helix transcription factor, has been suggested as playing multiple roles in mesoderm formation and chondrogenesis (Brown et al., Development, 126: 4317-29 (1999); Schweitzer et al., Development, 128: 3855-66 (2001)). Runx2/Cbfa1 is an osteopath specific transcription factor.

MCLs of young adult male rats were excised and the cells cultured for experimentation as in Example 1. Total RNA was isolated using the TRI reagent (Molecular Research Center, Inc., Cincinnati, Ohio) following the manufacturer's recommendation. The Six1 probe was purchased from ATCC. Probes for scleraxis, Runx2/Cbfa1, and type I collagen were obtained by PCR. The cDNA probes for ActR-I, BMPR-IA, BMPR-IB, and BMPR-II were obtained by digestion of the corresponding plasmids with the appropriate restriction endonucleases according to Yeh et al., J. Cell Physiol. 185: 87-97 (2000). The cDNA probes were labeled with ³² P-DATP using the Strip-EZ labeling kit from Ambion (Austin, Tex.). The Northern analyses were conducted as described in Yeh et al., Endocrinology 138: 4181-90 (1997).

Messenger RNA expression of control cells and cells treated with 200 ng/ml of OP-1 was measured over 16 days. Total RNAs (20 μg) were denatured and fractionated on 1% GTG agarose gels containing 2.2 M formaldehyde. The fractionated RNA was transferred onto a “Nytran Plus” membrane using a Turboblot apparatus (Schleicher & Schuell, Inc., Keene, N.H.) and was covalently linked to the membrane using the UV Crosslinker (Stratagene, La Jolla, Calif.). The membranes were incubated overnight at 42° C. with cDNA probes, washed, exposed to screen for the PhosphorImager (Molecular Dynamics, Sunnyvale, Calif.), and analyzed. Before probing with another probe, the blots were stripped at 68° C. with the Strip-EZ Probe Degradation Buffer (Ambion, Austin, Tex.) according to the protocol of the manufacturer and checked to ensure that the level of radioactivity was reduced to background. The blots were also probed with an 18S rRNA oligonucleotide to correct for loading variations.

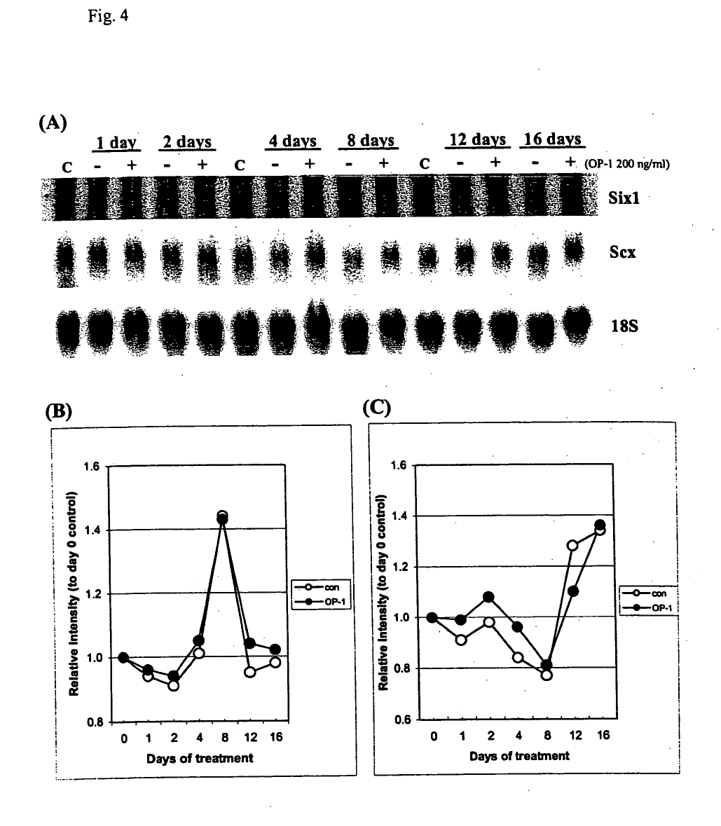

Control MCL cells expressed Six1 mRNA in a time-dependent manner, with a peak expression occurring at 8 days, returning to the control value afterwards. OP-1 treatment did not change the pattern of expression (see FIGS. 4A and 4B).

Control MCL cells expressed the scleraxis gene constitutively in a time-dependant manner. The expression level remained unchanged for the initial phase, but increased dramatically beginning at day 12. OP-1 treatment did not change its pattern of expression (see FIGS. 4A and 4C).

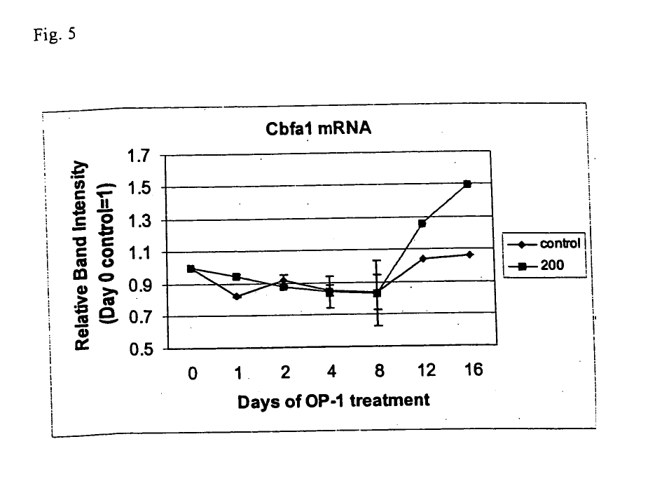

Messenger RNA coding for Runx2/Cbfa1 was detected in control MCL cells and the level was low for the entire 16 days of culture. OP-1 treatment did not change the Run2x/Cbfa1 mRNA level for the first 8 days, but increased it by about 1.5 fold thereafter (see FIG. 5).

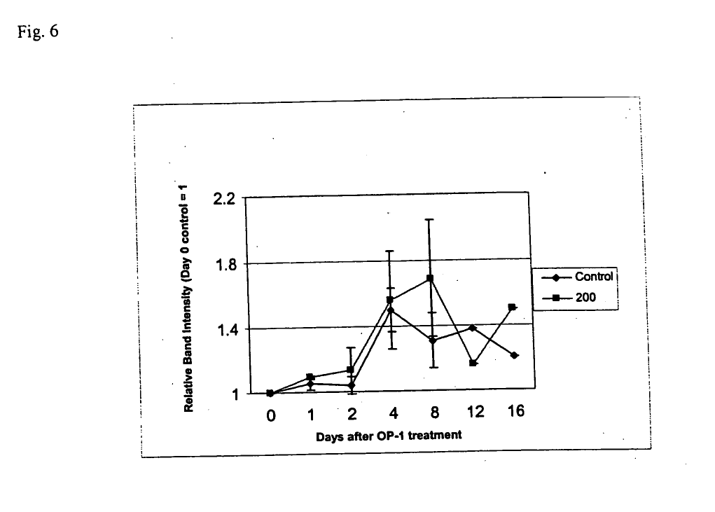

Control MCL cells expressed a high level of type I collagen mRNA. The basal mRNA level increased beginning about day 4 and remained elevated through day 16 in culture. The OP-1-treated MCL cells expressed a moderately elevated steady-state mRNA expression level in a time-dependent manner and reached a peak at about day 8, with an increase of about 30%. The level decreased gradually but was significantly higher than that of the control cells at day 16 (see FIG. 6).

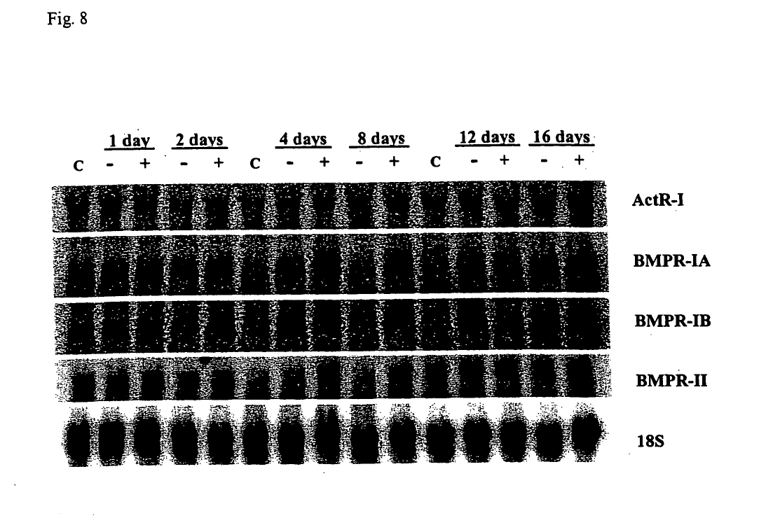

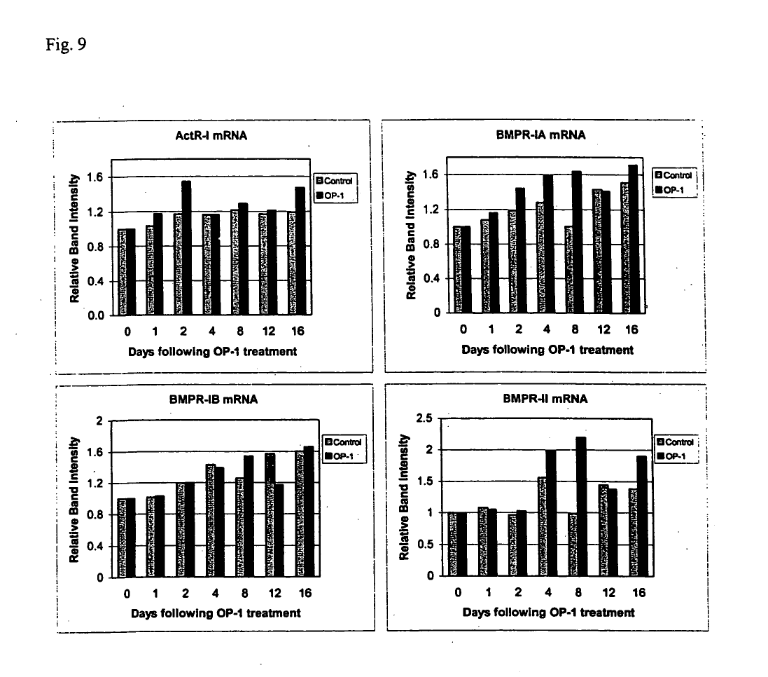

As Northern blot analysis demonstrated, MCL cells expressed the genes coding for ActR-I, BMPR-IA, BMPR-IB, and BMPR-II during the 16 days in culture. In the control cells, the ActR-I mRNA level increased slightly as a function of time (see FIG. 9). The BMPR-IA and BMPR-IB mRNA levels in control cells increased gradually and more substantially than the ActR-I mRNA level (see FIGS. 8 and 9). The BMPR-II mRNA level remained at the base level during the first 4 days in culture, but increased significantly thereafter (see FIGS. 8 and 9). OP-1 treatment did not significantly affect the ActR-I or BMPR-IB mRNA levels. OP-1 treatment increased the BMPR-IA mRNA level, with a maximum increase of about 60% over control on day 8 (see FIGS. 8 and 9). OP-1 treatment increased the BMPR-II mRNA level, with a maximum increase of about 100% over control (see FIGS. 8 and 9).

### Example 4

### Promoter Activity of Type-I Collagen Control and OP-1-Treated Rat MCL Cells

MCLs of young adult male rats were excised and the cells cultured for experimentation as in Example 1. A 1.372-kb DNA fragment, comprised of nucleotides from −1263 bp upstream to +109 bp downstream from the transcription start site (+1) of the rat type I collagen gene was generated by PCR using genomic DNA isolated from rat liver. The (−1263/+109) (SEQ. ID NO:11) promoter fragment was subcloned into pGL2-Basic vector (Promega Corp.) containing the promoterless luciferase report gene (Luc). A deletion clone (−1263(Δ−1026/−411)/+109) (SEQ. ID NO:12) was also generated by digestion of the parent plasmid with unique restriction enzymes Bal I (Msc I) followed by re-ligation. Both clones were confirmed by restriction enzyme mapping and double-stranded DNA sequencing. Primary cultures of rat MCL cells were transiently transfected with the type I collagen promoter constructs and treated with 50 or 200 ng/ml of OP-1 for 6 days. Luciferase activity was then measured and normalized to the β-galactosidase activity using the Dual assay kit (Tropix, Bedford, Mass.).

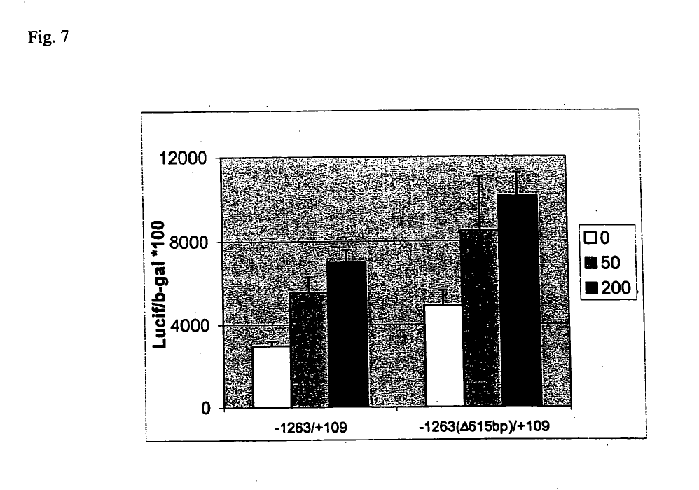

OP-1 stimulated the promoter activity of type I collagen in a dose-dependent manner. OP-1 stimulated the basal luciferase activity by about 15%. Clones containing the −1263/+109 and the −1263(Δ−1026/−411)/+109 promoter sequence treated with 50 ng/ml of OP-1 showed ˜80% increase in promoter activity. Those treated with 200 ng/ml of OP-1 showed about 140% increase in promoter activity (see FIG. 7).

### Example 5

### BMP mRNA Expression in Control and OP-1-Treated Rat MCL Cells

MCLs of young adult male rats were excised and the cells cultured for experimentation as in Example 1.

The mRNA expression of several BMPs in control and OP-1-treated MCL cultures was measured over 16 days using the RiboQuant RNase protection analysis (“RPA”) kit with a Mouse Multi-Probe Template Sets from BD Pharmingen (San Diego, Calif.). The mBMP-1 Multi-Probe Template Set permits detection of mRNAs for BMP-1, -2, -3, -4, -5, -6, -7, -8A and -8B. The protected fragments for BMP-1, -2, -3, -4, -5, -6, -7, -8A and -8B were 148, 160, 181, 226, 253, 283, 316, 353, and 133 nucleotides in length, respectively. The Template Set allows detection of mRNAs for ribosomal protein L32 and GAPDH allowing normalization of sampling or technique errors. The anti-sense RNA probes were labeled with ³²P-UTP using the RiboQuant in vitro transcription kit from BD PharMingen (San Diego, Calif.). The protected fragments were analyzed on 5% polyacrylamide gels containing 8M urea, detected using the PhosphorImager and quantified using the ImageQuant software (Molecular Dynamics, Sunnyvale Calif.).

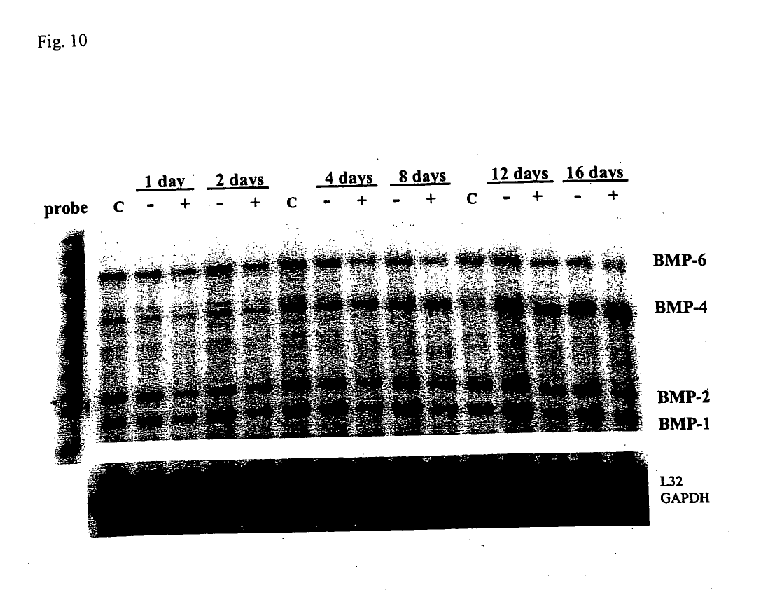

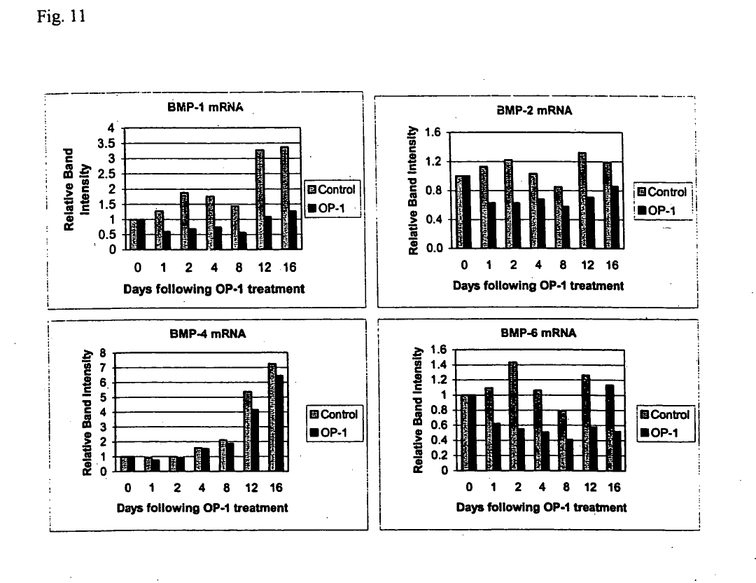

Significant levels of BMP-1, -2, -4, and -6 mRNA were detected in the control MCL cultures. As shown in FIGS. 10 and 11, the BMP-1 and BMP-4 mRNA levels increased as a function of time in the control cells. The BMP-1 mRNA level reached a maximum of about three times the day 0 level at day 16 in culture. The BMP-4 mRNA level increased dramatically as a function of time, reaching a maximum of about seven times the day 0 level at day 16 in culture. BMP-1 mRNA levels in OP-1-treated cells was lowered to approximately that of day 0 control throughout the entire 16 days. BMP-4 mRNA levels were not altered in OP-1-treated cells. The BMP-2 and BMP-6 mRNA levels changed slightly in a time-dependent, cyclical manner in control cells during the 16 days. OP-1 treatment resulted in a decrease of 20-40% of the BMP-2 mRNA levels when compared to control. OP-1 treatment reduced BMP-6 mRNA expression by as much as 50% when compared to control (see FIGS. 10 and 11).

### Example 6

### GDF mRNA Expression in Control and OP-1-Treated Rat MCL Cells

MCLs of young adult male rats were excised and the cells cultured for experimentation as in Example 1.

GDF levels were measured over 16 days using the RiboQuant RPA kit with a Mouse Multi-Probe Template Sets from BDPharmingen (San Diego, Calif.) as described in Example 5. The mGDF-1 Multi-Probe Template Set permits detection of GDF-1, -3, -5, -6, -8, and -9. The protected fragments for GDF-1, -3, -5, -6, -8, and -9 were 148, 160, 181, 226, 253, 283, and 316 nucleotides in length, respectively. The Template Set allows detection of mRNAs for ribosomal protein L32 and GAPDH allowing normalization of sampling or technique errors. The anti-sense RNA probes were labeled with ³²P-UTP using the RiboQuant in vitro transcription kit from BD PharMingen (San Diego, Calif.). The protected fragments were analyzed on 5% polyacrylamide gels containing 8M urea, detected using the PhosphorImager and quantified using the ImageQuant software (Molecular Dynamics, Sunnyvale Calif.).

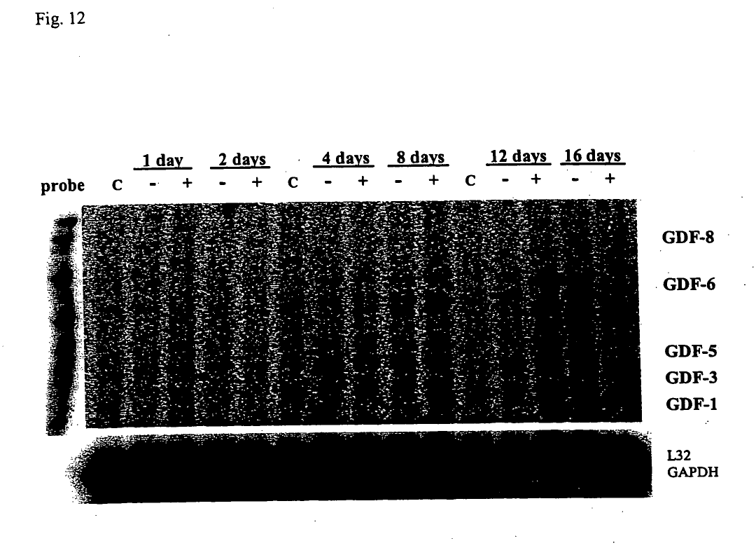

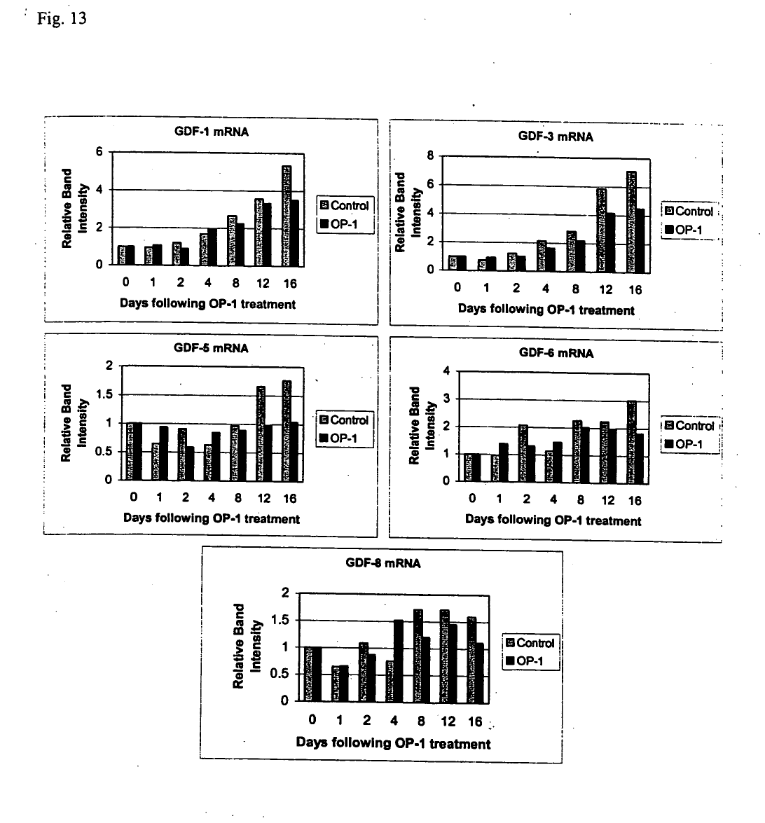

During the 16 days of culturing GDP-1 mRNA levels increased in both the control and OP-1-treated cells as a function of time reaching a maximum of about 5- and 3-fold, respectively, above the day 0 control. Similarly, GDF-3, -6, and -8 mRNA levels in control cells increased as a function of time, reaching a maximum of about 7-, 3-, and 1.7-fold, respectively, compared to day 0 control. OP-1 treatment lowered the extent of the increase without abolishing the time-dependent changes with a maximum of 4-, 2-, and 1.5-fold, respectively, compared to day 0 control. GDF-5 mRNA levels in control cultures increased to about 1.7-fold on day 8 as compared to day 0 control. OP-1 suppressed the increase except for day 4 (see FIGS. 12 and 13).

### Example 7

### Culturing Ligament Tissue Ex-Vivo

MCLs are surgically excised from the surrounding connective tissues at the knee joint of a patient under aseptic conditions. The MCLs are rinsed with HBSS plus penicillin-streptomycin (100 units/ml penicillin and 100 mg/ml streptomycin), and cut into small pieces.

The ligament pieces are cultured in DMEM/F12 (1:1) medium with 10% FBS supplemented with 30 μg/ml of gentamicin at 37° C. with 5% CO₂. Cells will begin to emerge from the tissue pieces and attach to the surface of the culture dishes after 3-4 days in culture. After 6-7 days, the tissue pieces are removed and the attached cells are cultured in fresh media until confluent.

The ligament cells are detached from the culture dishes by treatment with a mixture of trypsin-EDTA for 1 to 2 min or until all cells are detached. Cells are subcultured until confluent and frozen in liquid N₂ and revived for treatment. Cells are revived from the frozen stock in 100 mm or 150 mm dishes until confluent and subcultured at a cell density of 4×10⁴ cells/ml.

Cultured ligament cells are treated with an osteogenic protein (e.g. OP-1) in serum-free media for a pre-determined time period, harvested by trypsin-EDTA treatment, washed with media, and suspended in sterile HBSS for implantation into the patient.

### Example 8

### Implantation into Animal

Male rats will undergo surgery. After general anesthesia, the rats will be placed in a supine position and the knee joint will be exposed. A full thickness ligament defect will be created in the ACL. The animals will be divided into four groups. The defect in the first group of animals (the control group) will be treated with buffer or vehicle. The defect in the second group of animals will be treated with OP-1 (5-5000 ng/ml). The defect in the third group of animals will be treated with ligament cells that have been cultured ex-vivo and treated with OP-1 (5-5000 ng/ml). The defect in the fourth group of animals will be treated with ligament cells that have been cultured ex-vivo in the presence of OP-1 (5-5000 ng/ml); and treated with OP-1 (5-5000 ng/ml). In all cases, the joint will then be closed and sutured. The animals will be allowed to recover from anesthesia. After 4, 8 and 12 weeks, the animals will be euthanized and the ACL examined.

1. A method for treating a ligament defect in a patient comprising the steps of:
(a) isolating ligament cells;
(b) culturing the ligament cells ex-vivo;
(c) recovering the cultured ligament cells; and
(d) implanting the recovered ligament cells into the patient.

2. The method of claim 1 further comprising the step of administering to the patient a therapeutically effective amount of a bone morphogenic protein.

3. The method of claim 1 further comprising the step of transfecting the cultured ligament cells with a nucleic acid sequence encoding a bone morphogenic protein or a growth factor.

4. The method of claim 1 or 2 further comprising the step of treating the cultured ligament cells with a bone morphogenic protein.

5. The method of claim 1 or 2 further comprising the step of culturing the ligament cells for a time sufficient to allow formation of a ligament cell-associated matrix.

6. The method of claim 5, wherein the ligament cell-associated matrix is selected from the group consisting of type 1 collagen, elastin, decorin and aggrecan.

7. The method of any one of claims 1-5, wherein the bone morphogenic protein is selected from the group consisting of OP-1, OP-2, OP-3, BMP-2, BMP-3, BMP-4, BMP-5, BMP-6, BMP-8, BMP-9, BMP-10, BMP-11, BMP-15, BMP-16, DPP, Vg1, Vgr-1, 60A protein, GDF-1, GDF-2, GDF-3, GDF-5, GDF-6, GDF-7, GDF-8, GDF-9, GDF-10, GDF-11, GDF-12, NODAL, UNIVIN, SCREW, ADMP, NEURAL, and amino acid sequence variants thereof.

8. The method of any one of claims 1-5, wherein the bone morphogenic protein comprises an amino acid sequence having at least 70% homology with the C-terminal 102-106 amino acids, including the conserved seven cysteine domain of human OP-1, said bone morphogenic protein being capable of treating the ligament defect.

9. The method of any one of claims 1-5, wherein the bone morphogenic protein is selected from the group consisting of OP-1, GDF-5, GDF-6 and GDF-7.

10. The method of any one of claims 1-5, wherein the bone morphogenic protein is OP-1.

11. A method of repairing a ligament defect in a patient comprising the steps of:
(a) isolating ligament cells;
(b) culturing the ligament cells ex-vivo;
(c) recovering the cultured ligament cells; and
(d) implanting the recovered ligament cells into the patient.

12. The method of claim 11 further comprising the step of administering to the patient a therapeutically effective amount of a bone morphogenic protein.

13. The method of claim 11 further comprising the step of transfecting the cultured ligament cells with a nucleic acid sequence encoding a bone morphogenic protein or a growth factor.

14. The method of claim 11 or 12 further comprising the step of treating the cultured ligament cells with a bone morphogenic protein.

15. The method of claim 11 or 12 further comprising the step of culturing the ligament cells for a time sufficient to allow formation of a ligament cell-associated matrix.

16. The method of claim 15, wherein the ligament cell-associated matrix is selected from the group consisting of type 1 collagen, elastin, decorin and aggrecan.

17. The method of any one of claims 11-15, wherein the bone morphogenic protein is selected from the group consisting of OP-1, OP-2, OP-3, BMP-2, BMP-3, BMP-4, BMP-5, BMP-6, BMP-8, BMP-9, BMP-10, BMP-11, BMP-15, BMP-16, DPP, Vg1, Vgr-1, 60A protein, GDF-1, GDF-2, GDF-3, GDF-5, GDF-6, GDF-7, GDF-8, GDF-9, GDF-10, GDF-11, GDF-12, NODAL, UNIVIN, SCREW, ADMP, NEURAL, and amino acid sequence variants thereof.

18. The method of any one of claims 11-15, wherein the bone morphogenic protein comprises an amino acid sequence having at least 70% homology with the C-terminal 102-106 amino acids, including the conserved seven cysteine domain of human OP-1, said bone morphogenic protein being capable of treating the ligament defect.

19. The method of any one of claims 11-15, wherein the bone morphogenic protein is selected from the group consisting of OP-1, GDF-5, GDF-6 and GDF-7.

20. The method of any one of claims 11-15, wherein the bone morphogenic protein is OP-1.

21. A method of regenerating ligament tissue in a patient comprising the steps of:
(a) isolating ligament cells;
(b) culturing the ligament cells ex-vivo;
(c) recovering the cultured ligament cells; and
(d) implanting the recovered ligament cells into the patient.

22. The method of claim 21 further comprising the step of administering to the patient a therapeutically effective amount of a bone morphogenic protein.

23. The method of claim 21 further comprising the step of transfecting the cultured ligament cells with a nucleic acid sequence encoding a bone morphogenic protein or a growth factor.

24. The method of claim 21 or 22 further comprising the step of treating the cultured ligament cells with a bone morphogenic protein.

25. The method of claim 21 or 22 further comprising the step of culturing the ligament cells for a time sufficient to allow formation of a ligament cell-associated matrix.

26. The method of claim 25, wherein the ligament cell-associated matrix is selected from the group consisting of type 1 collagen, elastin, decorin and aggrecan.

27. The method of any one of claims 21-25, wherein the bone morphogenic protein is selected from the group consisting of OP-1, OP-2, OP-3, BMP-2, BMP-3, BMP-4, BMP-5, BMP-6, BMP-8, BMP-9, BMP-10, BMP-11, BMP-15, BMP-16, DPP, Vg1, Vgr-1, 60A protein, GDF-1, GDF-2, GDF-3, GDF-5, GDF-6, GDF-7, GDF-8, GDF-9, GDF-10, GDF-11, GDF-12, NODAL, UNIVIN, SCREW, ADMP, NEURAL, and amino acid sequence variants thereof.

28. The method of any one of claims 21-25, wherein the bone morphogenic protein comprises an amino acid sequence having at least 70% homology with the C-terminal 102-106 amino acids, including the conserved seven cysteine domain of human OP-1, said bone morphogenic protein being capable of regenerating ligament tissue.

29. The method of claim any one of claims 21-25, wherein the bone morphogenic protein is selected from the group consisting of OP-1, GDF-5, GDF-6 and GDF-7.

30. The method of claim any one of claims 21-25, wherein the bone morphogenic protein is OP-1.

31. A method of forming ligament tissue in a patient comprising the steps of:
(a) isolating ligament cells;
(b) culturing the ligament cells ex-vivo;
(c) recovering the cultured ligament cells; and
(d) implanting the recovered ligament cells into the patient.

32. The method of claim 31 further comprising the step of administering to the patient a therapeutically effective amount of a bone morphogenic protein.

33. The method of claim 31 further comprising the step of transfecting the cultured ligament cells with a nucleic acid sequence encoding a bone morphogenic protein or a growth factor.

34. The method of claim 31 or 32 further comprising the step of treating the cultured ligament cells with a bone morphogenic protein.

35. The method of claim 31 or 32 further comprising the step of culturing the ligament cells for a time sufficient to allow formation of a ligament cell-associated matrix.

36. The method of claim 35, wherein the ligament cell-associated matrix is selected from the group consisting of type 1 collagen, elastin, decorin and aggrecan.

37. The method of any one of claims 31-35, wherein the bone morphogenic protein is selected from the group consisting of OP-1, OP-2, OP-3, BMP-2, BMP-3, BMP-4, BMP-5, BMP-6, BMP-8, BMP-9, BMP-10, BMP-11, BMP-15, BMP-16, DPP, Vg1, Vgr-1, 60A protein, GDF-1, GDF-2, GDF-3, GDF-5, GDF-6, GDF-7, GDF-8, GDF-9, GDF-10, GDF-11, GDF-12, NODAL, UNIVIN, SCREW, ADMP, NEURAL, and amino acid sequence variants thereof.

38. The method of any one of claims 31-35, wherein the bone morphogenic protein comprises an amino acid sequence having at least 70% homology with the C-terminal 102-106 amino acids, including the conserved seven cysteine domain of human OP-1, said bone morphogenic protein being capable of forming ligament tissue.

39. The method of any one of claims 31-35, wherein the bone morphogenic protein is selected from the group consisting of OP-1, GDF-5, GDF-6 and GDF-7.

40. The method of any one of claims 31-35, wherein the bone morphogenic protein is OP-1.

41. A method of promoting ligament tissue formation in a patient comprising the steps of:
(a) isolating ligament cells;
(b) culturing the ligament cells ex-vivo;
(c) recovering the cultured ligament cells; and
(d) implanting the recovered ligament cells into the patient.

42. The method of claim 41 further comprising the step of administering to the patient a therapeutically effective amount of a bone morphogenic protein.

43. The method of claim 41 further comprising the step of transfecting the cultured ligament cells with a nucleic acid sequence encoding a bone morphogenic protein or a growth factor.

44. The method of claim 41 or 42 further comprising the step of treating the cultured ligament cells with a bone morphogenic protein.

45. The method of claim 41 or 42 further comprising the step of culturing the ligament cells for a time sufficient to allow formation of a ligament cell-associated matrix.

46. The method of claim 45, wherein the ligament cell-associated matrix is selected from the group consisting of type 1 collagen, elastin, decorin and aggrecan.

47. The method of any one of claims 41-45, wherein the bone morphogenic protein is selected from the group consisting of OP-1, OP-2, OP-3, BMP-2, BMP-3, BMP-4, BMP-5, BMP-6, BMP-8, BMP-9, BMP-10, BMP-11, BMP-15, BMP-16, DPP, Vg1, Vgr-1, 60A protein, GDF-1, GDF-2, GDF-3, GDF-5, GDF-6, GDF-7, GDF-8, GDF-9, GDF-10, GDF-11, GDF-12, NODAL, UNIVIN, SCREW, ADMP, NEURAL, and amino acid sequence variants thereof.

48. The method of any one of claims 41-45, wherein the bone morphogenic protein comprises an amino acid sequence having at least 70% homology with the C-terminal 102-106 amino acids, including the conserved seven cysteine domain of human OP-1, said bone morphogenic protein being capable of promoting ligament tissue formation.

49. The method of any one of claims 41-45, wherein the bone morphogenic protein is selected from the group consisting of OP-1, GDF-5, GDF-6 and GDF-7.

50. The method of any one of claims 41-45, wherein the bone morphogenic protein is OP-1.

51. The method of any one of claims 7, 17, 27, 37, or 47 wherein the bone morphogenic protein is formulated with a carrier.

52. The method of claim 51, wherein the carrier is selected from the group consisting of collagen, hydroxyapatite, carboxymethyl cellulose, tricalcium phosphate, polylactic acid, polybutyric acid and polyglycolic acid.

53. A composition comprising cultured ligament cells and a bone morphogenic protein.

54. The composition of claim 53 further comprising a ligament cell-associated matrix.

55. The composition of claim 54, wherein the ligament cell-associated matrix is selected from the group consisting of type 1 collagen, elastin, decorin and aggrecan.

56. The composition of claim 53 or 54, wherein the bone morphogenic protein is selected from the group consisting of OP-1, OP-2, OP-3, BMP-2, BMP-3, BMP-4, BMP-5, BMP-6, BMP-8, BMP-9, BMP-10, BMP-11, BMP-15, BMP-16, DPP, Vg1, Vgr-1, 60A protein, GDF-1, GDF-2, GDF-3, GDF-5, GDF-6, GDF-7, GDF-8, GDF-9, GDF-10, GDF-11, GDF-12, NODAL, UNIVIN, SCREW, ADMP, NEURAL, and amino acid sequence variants thereof.

57. The composition of claim 53 or 54, wherein the bone morphogenic protein comprises an amino acid sequence having at least 70% homology with the C-terminal 102-106 amino acids, including the conserved seven cysteine domain of human OP-1, said bone morphogenic protein being capable of treating a ligament defect.

58. The composition of claim 53 or 54, wherein the bone morphogenic protein is selected from the group consisting of OP-1, GDF-5, GDF-6 and GDF-7.

59. The composition of claim 53 or 54, wherein the bone morphogenic protein is OP-1.

60. The composition of claim 53 or 54, wherein the ligament cells are transfected with a nucleic acid sequence encoding a bone morphogenic protein or a growth factor.
